(19) United States
(12) Patent Application Publication
(10) Pub. No.: US 2014/0024100 A1
(43) Pub. Date: Jan. 23, 2014

(54) MEASLES VIRUSES WITH REDUCED SUSCEPTIBILITY TO NEUTRALIZATION

(71) Applicant: Mayo Foundation for Medical Education and Research, Rochester, MN (US)

(72) Inventors: Stephen James Russell, Rochester, MN (US); Patrycja Lech, Rochester, MN (US)

(21) Appl. No.: 13/946,430

(22) Filed: Jul. 19, 2013

Related U.S. Application Data
(60) Provisional application No. 61/674,185, filed on Jul. 20, 2012.

(51) Int. Cl. C07K 14/005; C12N 7/00

(52) U.S. Cl. CPC C07K 14/005; C12N 7/00
USPC 435/235.1; 530/350; 536/23.72

(57) ABSTRACT

This document provides methods and materials for making and using measles viruses having a reduced susceptibility to antibody neutralization (e.g., antibody neutralization by monoclonal anti-measles virus antibodies and/or serum from measles virus vaccinees). For example, H polypeptides having a reduced ability of being recognized by anti-measles virus antibodies that were generated against a wild-type measles virus or a pre-existing measles virus vaccine such as MV-Edm as compared to a wild-type measles virus H polypeptide or the H polypeptide of MV-Edm are provided.

### CROSS-REFERENCE TO RELATED APPLICATIONS

This application claims the benefit of U.S. Provisional Application Ser. No. 61/674,185, filed Jul. 20, 2012. The disclosure of the prior application is considered part of (and is incorporated by reference in) the disclosure of this application.

### BACKGROUND

1. Technical Field

This document relates to methods and materials for making and using measles viruses having a reduced susceptibility to antibody neutralization (e.g., antibody neutralization by serum from measles virus vaccinees).

2. Background Information

Measles virus (MV) caused approximately 139,300 deaths in 2010, mostly amongst children under the age of five. Unvaccinated children are at highest risk of measles and measles related deaths. In particular, infants whose maternal anti-measles antibody titers have waned to non-protective levels, but are still too young to receive the current measles vaccine recommended for infants at 9-12 months, can be at an elevated risk of measles and measles related deaths.

Measles often is accompanied by immune suppression, which is thought to contribute to the susceptibility to secondary infections that account for most of the morbidity and mortality associated with the disease (Borrow and Oldstone, Curr Top. Microbiol. Immunol., 191:85-100 (1995)). A live attenuated strain of the measles virus, MV-Edm, has been used to vaccinate against the disease and its sequelae (Duclos and Ward, Drug Saf., 19:435-454 (1998)).

The measles virus is serologically monotypic. Life-long immunity is conferred by a single attack of measles or following vaccination with the measles virus vaccine. Despite genetic variation of circulating wild-type genotypes, it appears that none have escaped neutralization by serum from vaccinees since the introduction of the vaccine nearly 60 years ago.

### SUMMARY

This document provides methods and materials for making and using measles viruses having a reduced susceptibility to antibody neutralization (e.g., antibody neutralization by monoclonal anti-measles virus antibodies and/or serum from measles virus vaccinees). For example, this document provides H polypeptides having a reduced ability of being recognized by anti-measles virus antibodies that were generated against a wild-type measles virus or a pre-existing measles virus vaccine of genotype A (i.e., a measles virus vaccine of AIK-C, Moraten, Rubeovax, Schwarz, Zagreb, CAM-70, Changchun-47, Leningrad-4, Leningrad 16, or Shanghai-191) as compared to a wild-type measles virus H polypeptide or the H polypeptide of a pre-existing measles virus vaccine of genotype A. This document also provides (a) nucleic acid molecules encoding such H polypeptides, (b) measles virus particles having a reduced susceptibility to antibody neutralization (e.g., antibody neutralization by monoclonal anti-measles virus antibodies and/or serum from measles virus vaccinees such as those vaccinated with a pre-existing measles virus vaccine of genotype A), and (c) compositions containing such measles virus particles. For example, this document provides compositions containing measles viruses that have a reduced susceptibility (as compared to a wild-type measles virus or a pre-existing measles virus vaccine of genotype A) to being neutralized by anti-measles virus antibodies that were generated against a wild-type measles virus or a pre-existing measles virus vaccine of genotype A. In some cases, this document provides non-measles viruses (e.g., retroviruses such as lentivirus or gammaretrovirus) that contain a measles virus H polypeptide having a reduced ability (as compared to a wild-type measles virus H polypeptide or the H polypeptide of a pre-existing measles virus vaccine of genotype A) of being recognized by anti-measles virus antibodies that were generated against a wild-type measles virus or a pre-existing measles virus vaccine of genotype A.

In addition, this document provides methods for treating cancer. For example, a measles or non-measles virus containing a modified H polypeptide described herein can be used as a therapeutic agent for the treatment of cancer (e.g., ovarian cancer, breast cancer, or glioma) in a manner that allows the virus to exert its therapeutic effect with reduced susceptibility to being neutralized by anti-measles virus antibodies existing within the cancer patient. Such anti-measles virus antibodies can be those that the cancer patient developed years earlier during a standard measles virus vaccination such as a vaccination involving use of a measles virus vaccine of genotype A or a natural measles virus infection.

This document also provides methods for using a measles virus preparation provided herein to vaccinate infants (e.g., infants less than 9 or less than 15 months of age) against measles virus infections at a time when they may potentially have maternal anti-measles virus antibodies capable of neutralizing standard measles virus vaccines such as pre-existing measles virus vaccines of genotype A. For example, a measles virus containing a modified H polypeptide provided herein can be used as a vaccine in a manner that allows the measles virus of the vaccine to exert its immunogenic effect with reduced susceptibility to being neutralized by maternal anti-measles virus antibodies existing within the infant. Such maternal anti-measles virus antibodies can be those that the infant's mother developed years earlier during a standard measles virus vaccination such as a vaccination involving use of a pre-existing measles virus vaccine of genotype A or a natural measles virus infection.

Viruses (e.g., measles viruses and non-measles viruses) containing a wild-type measles virus H polypeptide can be neutralized within a mammal by anti-measles virus antibodies that were generated when the mammal was vaccinated with a pre-existing measles virus vaccine of genotype A or infected naturally with a naturally occurring measles virus. The measles virus H polypeptides provided herein can be heterologous to naturally occurring measles virus H polypeptides (e.g., heterologous to an H polypeptide having a sequence set forth in SEQ ID NO:2 or SEQ ID NO:4) and can contain amino acid sequence substitutions as compared to a naturally occurring H polypeptide. As a result of these amino acid substitutions, a measles virus H polypeptide provided herein can have less susceptibility to antibody neutralization within a mammal by anti-measles virus antibodies that were generated when the mammal was vaccinated with a pre-existing measles virus vaccine of genotype A or infected naturally with a naturally occurring measles virus than that of a naturally occurring measles virus H polypeptide.

As described herein, this document provides viruses (e.g., measles viruses and non-measles viruses) containing a nucleic acid molecule encoding a modified H polypeptide. Typically, such viruses will express the modified H polypeptide and incorporate it into the virus particle. Because the modified viral H polypeptides can have less susceptibility to antibody neutralization within a mammal by anti-measles virus antibodies than naturally occurring H polypeptides, viruses containing the modified H polypeptides can have less susceptibility to such antibody neutralization. Reduced susceptibility to such antibody neutralization can result in increased effectiveness of the virus when the virus is administered to a mammal (e.g., to a cancer patient to be treated using the virus as an anti-cancer agent or to an infant to be vaccinated against measles virus infections using the virus as an immunogenic agent). For example, a virus provided herein can be used to treat cancer patients without being neutralized prior to exerting its anti-cancer effect.

This document is based, in part, on the development of modified measles virus H polypeptides that can be incorporated into virus particles and exhibit a reduced susceptibility to antibody neutralization by anti-measles virus antibodies that were generated against a wild-type measles virus or a pre-existing measles virus vaccine of genotype A.

In general, one aspect of this document features a measles virus H polypeptide comprising, or consisting essentially of, the amino acid sequence set forth in SEQ ID NO:2 or SEQ ID NO:4 with the exception that the measles virus H polypeptide comprises an amino acid substitution at three or more of the positions selected from the group consisting of L284, Y310, E395, E398, K403, N405, D416, K488, E489, A490, E535, H536, A537, S546, R547, S550, F552, Y553, P554, S590, G592, W472, P474, and G316. In some cases, measles viruses comprising the measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 ascites/well of 16CD-11, 10 μg/well of BH18, 6μL ascites/well of BH47, or 50 hybridoma supernatant of 20H6, when the measles viruses are placed into a well of a 96-well microtiter plate. In some cases, measles viruses comprising the measles virus H polypeptide are not neutralized by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (1) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when the measles viruses are placed into a well of a 96-well microtiter plate.

In another aspect, this document features a measles virus H polypeptide wherein measles viruses comprising the measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, or 50 μL hybridoma supernatant of 20H6, when the measles viruses are placed into a well of a 96-well microtiter plate. In some cases, measles viruses comprising the measles virus H polypeptide are not neutralized by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (l) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when the measles viruses are placed into a well of a 96-well microtiter plate. In some cases, measles viruses comprising the measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, and 50 μL hybridoma supernatant of 20H6, when the measles viruses are placed into a well of a 96-well microtiter plate.

In another aspect, this document features a nucleic acid encoding a measles virus H polypeptide set forth in either of the two preceding paragraphs. In another aspect, this document features a recombinant virus comprising such a nucleic acid. The virus can be a measles virus.

In another aspect, this document features a recombinant virus comprising a measles virus H polypeptide set forth in either of such two preceding paragraphs. The virus can be a measles virus.

In another aspect, this document features a recombinant measles virus wherein the measles virus is at least four times less sensitive than MV-Edm to neutralization by serum from a measles-vaccinated human.

In another aspect, this document features a recombinant measles virus wherein the measles virus is at least four times less sensitive than MV-Edm to neutralization by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (1) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when the measles virus is placed into a well of a 96-well microtiter plate.

In another aspect, this document features a method for reducing the number of viable tumor cells in a mammal The method comprises, or consist essentially of, administering to the mammal a virus provided herein.

In another aspect, this document features a method for stimulating an immune response against measles virus in a human whose anti-MV antibody titer is greater than 6 mIU/mL. The method comprises, or consist essentially of, administering to the human a virus provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Rationally designed mutations delineate four different immunodominant epitopes of hemagglutinin protein recognized by neutralizing monoclonal antibodies. (A) MV-H14, (B) MV-H11, (C) MV-H5, and (D) MV-H22 were incubated in media (black bars) or neutralizing concentrations of BH15, 16DE6, I-41, c87, c148, I-44, BH141 and I-29 (gray bars) for 1.5 hours at 37° C. Infection of Vero cells was scored as the number of eGFP expressing syncytia per well, 48 hours post infection. Experiments were performed in a 96-well format in duplicate wells. Cartoon structures of the H cuboidal ectodomain (PDB 2ZB6) are shown as viewed downwards (Top View), illustrating the escape mutations in (E) MV-H11, (F) MV-H5, (G) MV-H22, and the mAbs they escape. The general location of epitope E1-4 is boxed and delineated by the location of the escape mutation(s) in each virus. All mutations are shown as spheres. Asparagines (N) available for N-linked glycosylation within a PNGS are highlighted. The circle illustrates a confirmed N-linked glycosylation. Highlighted spheres in (F) MV-H5 are mutated residues 395 and 398 for future reference. MV-H22 (G) does not escape *BH38, but previous studies localize it to E4 by virtue of a mutation in residue 310.

FIG. 2. MV-δE3 resists neutralization by monoclonal antibodies targeting E1-3. The ability of MV-δE3 to escape monoclonal antibodies recognizing E1-3 was assessed in Neutralization Assays. MV-eGFP, MV-H11, and MV-δE3 were incubated in the absence (black solid bars) or presence (hatched bars) of mAbs prior to infection of Vero cells. Infection was scored 48 hours later, as the number of eGFP expressing syncytia per well. Experiments were performed in a 96-well format in duplicate wells. Viruses were challenged with mAbs targeting E1 (A) and E2 and E3 (B, C) in different combinations. (D) Cartoon structure of MV-δE3 H cuboidal ectodomain illustrates all mutations as spheres. Asparagines (N) available for N-linked glycosylation in PNGS 282NDL→NDS and 535EHA→NAT are highlighted. Circles represent N-linked glycan shields. CL48 selected escape mutations E395K is labeled. E1-3 are delineated by the location of the escape mutations and are boxed with the mAbs that recognize them.

FIG. 3. MV-6E4 evades neutralization by a cocktail of mAbs targeting E1-4 simultaneously. (A) Cartoon structure of the MV-δE4 H cuboidal ectodomain (Top View) illustrates mutations as spheres. Asparagine (N) residues available for N-linked glycosylation in PNGS 282NDL→NDS and 535EHA→NAT are labeled. Circles represent glycan shields. c148 selected escape mutation E395K is highlight. BH38 selected mutation Y310C and also E471K (which was present in ¼ BH38 resistant clones sequenced) are highlighted. Box highlights E1-4, delineated by the location of mutations escaping mAbs present in each box. Underlined are mAbs used in the cocktail mix in (C) and (D). (B) MV-eGFP and MV-δE4 were incubated in the absence (black solid bar) and presence (hatched bars) of individual mAbs prior to infection of Vero cells. Infection was scored 48 hours later by counting the number of eGFP expressing syncytia per well. BH97 was used as a positive control for MV-δE4 neutralization. (C) MV-eGFP and MV-dE4 were then challenged with media alone (no mAb), a cocktail of mAbs targeting all four epitopes simultaneously: BH15 (E1), 16DE6 (E2), c148 (E3), and BH141 (E4) with or without BH97 (control). (D) Infection was visualized 48 hours post infection by fluorescence microscopy at 4x magnification.

FIG. 4. Escape mutations in MV-δE4 do not inhibit entry via cellular receptors CD46, SLAM, and Nectin-4, but they inhibit cell-cell fusion via Nectin-4 and SLAM. CHO cells or CHO cells stably expressing human CD46, SLAM, or Nectin-4 were infected with MV-eGFP or MV-6E4. Twenty four-hours post infection (A) fresh media or (B) media containing FIP was added to the cells. (A) The extent of cell-cell fusion following infection was imaged 72 hours post infection by fluorescent microscopy at 20× magnification. Mutations in MV-δE4 inhibited fusion via SLAM and Nectin-4, but not CD46. Infected CHO-Nectin-4 cells were imaged in phase to show the relative size of syncytia to single cells. (B) Cells were treated with FIP to inhibit fusion, and the relative number of infected cells was imaged 48 hours post infection. Escape mutations in MV-δE4 decrease the level of infection via SLAM and Nectin-4, but not CD46. CHO cells can be infected at a very low level. MV entry into CHO cells occurs via an unidentified receptor.

FIG. 5. Engineered escape mutations have varying effects on cell-cell fusion via MV cellular receptors: human CD46, SLAM, and Nectin-4. CHO cells (unmodified) and CHO cells transduced with either human CD46, SLAM, or NECTIN-4 were infected with MV-eGFP, MV-H7, MV-H8, MV-H11, MV-H14, MV-H16, MV-H21, MV-H20, MV-H22 (see Table 4), and MV-H.Shield at a moi of 0.1. Infected, eGFP expressing cells were visualized by fluorescent microscopy and images were taken at 20× magnification 72 hours post infection. Infections were performed in triplicate. If no eGFP expressing cells were visible 72 hours post infection in 3/3 wells, the image was taken in phase to show the presence of live cells. Very low levels of infection in CHO unmodified cells was anticipated as they facilitate very low levels of infection via an unidentified receptor.

FIG. 6. Engineered escape mutations have varying effects on infection via MV cellular receptors: human CD46, SLAM, and Nectin-4. CHO cells (unmodified) and CHO cells transduced with either human CD46, SLAM, or NECTIN-4 were infected with MV-eGFP, MV-H7, MV-H8, MV-H11, MV-H14, MV-H16, MV-H21, MV-H20, MV-H22 (see Table 4), and MV-H.Shield at a moi of 0.8. Sixteen hours post infection the virus containing media was replaced with media containing fusion inhibitory peptide (FIP) in order to visualize the number of infected cells in the absence of cell-cell fusion. Infected, eGFP expressing cells were visualized by fluorescent microscopy and images were taken at 20× magnification 48 hours post infection. Infections were performed in triplicate. If no eGFP expressing cells were visible 48 hours post infection in 3/3 wells, the image was taken in phase to show the presence of live cells. Very low levels of infection in CHO unmodified cells was anticipated as MV infects CHO cells at very low levels via an unidentified receptor.

FIG. 7. MV-H.Shield has significantly slower fusion kinetics than MV-eGFP. Vero cells were infected with MV-eGFP or MV-H.Shield at 500 PFU/mL in a 96 well format at 37° C. On Day 2 and Day 5 post infection, infected eGFP expressing cells were visualized by fluorescence microscopy. Images were taken at 4× magnification. On Day 2 post infection, MV-eGFP replication formed characteristic eGFP expressing syncytia, which expand and coalesced as the virus propagated causing the fused monolayer to lift off the plate by Day 5. MV-H.Shield displayed a non-fusogenic phenotype on Day 2 post infection. On Day 5, a retarded slow fusion phenotype became apparent.

FIG. 8. MV-H.Shield is modified by additional N-linked glycosylations. (A) Schematic depiction of the potential N-linked glycosylation sites (PNGS) encoded in the H.Shield gene. The H protein from the Edmonston-tag vaccine strain (genotype A) and its derivatives encodes five PNGS at N168, N187, N200, N215, and N238. Four of these sites are modified by a glycosylation as indicated. MV-H.Shield encodes an additional 6 PNGS (black arrows) of which four are confirmed to be N-linked glycosylated by western immunoblotting for viral H as indicated. (B) Immunoblot for measles virus H glycoprotiens. Engineered PNGS are listed in brackets next to each MV-H# and MV-H.Shield (see Table 4). Removal of N-linked glycosylations following PNGase F treatment resulted in a downward band shift in all MV H glycoprotiens. (C) PNGS 590NGS is not glycosylated but was still incorporated into H.Shield on the basis of mutations flanking and within the 590SGG triplet in genotype B2 and D7, respectively. Residues 590SGG in genotype A altered to generate PNGS 590NGS are underlined.

FIG. 9. MV-H.Shield resists neutralization by monoclonal antibodies targeting different epitopes. MV-H.Shield encodes escape mutations in 8 different epitopes recognized by monoclonal antibodies (see Table 4). MV-eGFP (control) and MV-H.Shield were incubated with mAbs targeting 6/8 epitopes. The epitope protected by N416 (E7) was not tested in this context but was shown to escape BH099. PNGS 590SGG→NGS was not glycosylated and as a result thought to be non-protective within this context. MV-H.Shield does not encode escape mutations against BH30 (E9) and BH47 (E10), and hence these mAb neutralize MV-H.Shield along with control MV-eGFP. Virus and mAbs were incubated for 1.5 hours at 37° C. prior to infection of Vero cells. The number of infected eGFP positive foci per well were counted 48 hours post infection and expressed as a percentage of the number of infected eGFP positive foci in the absence of mAb.

FIG. 10. MV-H.Shield is less susceptible to neutralization by pooled human serum than MV-eGFP. MV-eGFP and MV-H.Shield were subjected to a plaque reduction neutralization test (PRNT) to determine the dilution and titer of commercially available, heat inactivated, pooled human serum, that inhibited 50% (ND50; 50% neutralizing dose) and 100% (NT; neutralizing titer) infection of Vero cells. Virus and serum were incubated for (A) 2 hours at 37° C. and (B) 24 hours at 37° C. and 24 hours at 4° C. prior to infection of Vero cells. The number of infected eGFP expressing foci were counted 48 hours post infection under a fluorescent microscope.

FIG. 11. Tetrameric structure of MV-H in complex with SLAM (form II), highlighting escape mutations in H.Shield relative to receptor binding interface and dimer-of-dimers interface. (A) Surface, side view, representation for MV-H-SLAM tetramer complex (form II configuration) (PDB 3ALX). N-terminal stalk regions protruding from the viral envelope are schematically represented. Two H stalks dimerize via two disulphide bonds at the base of the cuboidal head. Within the tetramer each cuboidal head is in a different shade. SLAM (cartoon representation, black) marks the location of the receptor-binding interface. Point mutations—including those involved in PNGS—are highlighted. Pre-existing native glycosylated asparagines N200 and N215 are highlighted. N187 and N168 were not crystalized in this structure. Epitopes recognized by BH47 and BH30, not protected in H. Shield, are delineated by the location of the BH47 escape mutations L246S/S247P and BH30 escape mutation 472NIS. (B) Top View of the MV-H in complex with SLAM (form II). (C) Bottom View of the MV-H in complex with SLAM (form II).

FIG. 12 contains the nucleic acid sequence (SEQ ID NO:1) and amino acid sequence (SEQ ID NO:2) of a naturally occurring H polypeptide from the Edmonston MV strain (accession number K01711.1).

FIG. 13 contains the nucleic acid sequence (SEQ ID NO:3) and amino acid sequence (SEQ ID NO:4) of an H polypeptide from Edmonston-tag MV strain (accession number AB583749.1) into which the mutations described herein were introduced.

### DETAILED DESCRIPTION

This document provides nucleic acids, polypeptides, and viruses containing the nucleic acids and/or polypeptides. This document also provides methods for using the viruses to treat cancer patients or to vaccinate infants to help protect them from measles virus infections. For example, this document provides nucleic acid molecules encoding measles virus hemagglutinin (H) polypeptides, measles virus H polypeptides, and viruses containing such nucleic acids and/or such measles virus H polypeptides. The viruses described herein can be used to treat cancer patients or to vaccinate infants in a manner such that the viruses exhibit a reduced susceptibility to antibody neutralization.

### 1. Nucleic Acids

This document provides nucleic acid molecules that encode an H polypeptide that (1) is heterologous to naturally occurring H polypeptides, and (2) has reduced susceptibility to antibody neutralization as compared to a naturally occurring H polypeptide. The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

An “isolated nucleic acid” refers to a nucleic acid that is separated from other nucleic acid molecules that are present in a viral genome, including nucleic acids that normally flank one or both sides of the nucleic acid in a viral genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

A nucleic acid provided herein can encode a measles virus H polypeptide that is heterologous to naturally occurring measles virus H polypeptides or to the H polypeptide having the amino acid sequence set forth in SEQ ID NO:2. In some cases, a measles virus H polypeptide designed to be heterologous to naturally occurring measles virus H polypeptides and/or heterologous to the H polypeptide having the amino acid sequence set forth in SEQ ID NO:2 can be referred to as a modified H polypeptide. The term “H polypeptide amino acid sequence” as used herein refers to an amino acid sequence that is at least 85 percent (e.g., at least 85, 90, 95, 99, or 100 percent) identical to the sequence set forth in SEQ ID NO:2.

The percent identity between a particular amino acid sequence and the amino acid sequence set forth in SEQ ID NO:2 is determined as follows. First, the amino acid sequences are aligned using the BLAST 2 Sequences (B12seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the full-length H polypeptide amino acid sequence followed by multiplying the resulting value by 100. For example, an amino acid sequence that has 500 matches when aligned with the sequence set forth in SEQ ID NO:2 is 81.0 percent identical to the sequence set forth in SEQ ID NO:2 (i.e., 500÷617*100=81.0).

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

A nucleic acid molecule provided herein can encode a measles virus H polypeptide that contains one or more amino acid substitutions as compared to a naturally occurring measles virus H polypeptide or the measles virus H polypeptide having the amino acid sequence set forth in SEQ ID NO:2 or SEQ ID NO:4 such that measles viruses containing the encoded H polypeptide are less susceptible (e.g., two times, three times, four times, or more less susceptible) to antibody neutralization than measles viruses containing the corresponding, naturally occurring H polypeptide or measles viruses containing the measles virus H polypeptide having the amino acid sequence set forth in SEQ ID NO:2 or SEQ ID NO:4. For example, a nucleic acid molecule provided herein can encode a measles virus H polypeptide having the amino acid sequence set forth in SEQ ID NO:2 or SEQ ID NO:4 with the exception that the measles virus H polypeptide has an amino acid substitution at three or more (e.g., 3, 4, 5, 6, 7, 8, 9, 10, or more) of the following positions: P279, L284, Y310, K389, E395, E398, K403, N405, D416, K488, E489, A490, E535, H536, A537, S546, R547, S550, F552, Y553, P554, S590, G592, E471, W472, P474, and G316. In some cases, a nucleic acid molecule provided herein can encode a measles virus H polypeptide having the amino acid sequence set forth in SEQ ID NO:2 or SEQ ID NO:4 with the exception that the measles virus H polypeptide has three or more (e.g., 3, 4, 5, 6, 7, 8, 9, 10, or more) of the following amino acid substitutions: P279R, L284S, Y310C, K389R, E395K, E398G, K403N, N405T, D416N, K488N, E489A, A490T, E535N, H536A, A537T, S546G/R, R547A/C, S550T, F552N, Y553G, P554T, S590N, G592S, E471A, W472N, P474S, R547C, and G316N.

The ability of a measles virus to be less susceptible (e.g., two times, three times, four times, or five times less susceptible) to antibody neutralization refers to the ability of a measles virus to induce syncytia formation in the presence of anti-measles virus antibodies that were generated against a naturally occurring measles virus or pre-existing measles virus vaccine of genotype A or in the presence of one or more (e.g., two, three, four, five, six, seven, eight, or nine) of the following antibodies: a BH15 antibody, a 16DE6 antibody, a c87 antibody, a c118 antibody, a c8 antibody, a I-41 antibody, a c148 antibody, a I-44 antibody, a I-29 antibody, a BH38 antibody, a BH141 antibody, a 20H6 antibody, a BH97 antibody, a 20H6 antibody, a 16CD11 antibody, a BH099 antibody, a BH18 antibody, and/or a BH30 antibody. In some cases, a measles virus H polypeptide provided herein can be designed to have an amino acid sequence such that measles viruses containing that measles virus H polypeptide and no wild-type measles virus H polypeptides are capable of infection in the presence of anti-measles virus antibodies with a reduced susceptibility to antibody neutralization by those anti-measles virus antibodies. In such cases, the anti-measles virus antibodies can be anti-measles virus antibodies that were generated against a naturally occurring measles virus, anti-measles virus antibodies that were generated against a pre-existing measles virus vaccine of genotype A (e.g., a MV-Edm measles virus vaccine), a BH15 antibody, a 16DE6 antibody, a c87 antibody, a c118 antibody, a c8 antibody, a I-41 antibody, a c148 antibody, a I-44 antibody, a I-29 antibody, a BH38 antibody, a BH141 antibody, a 20H6 antibody, a BH97 antibody, a 20H6 antibody, a 16CD11 antibody, a BH099 antibody, a BH18 antibody and a BH30 antibody, or a combination thereof (e.g., a combination of a BH15 antibody, a 16DE6 antibody, a c87 antibody, a c118 antibody, a c8 antibody, a I-41 antibody, a c148 antibody, a I-44 antibody, a I-29 antibody, a BH38 antibody, a BH141 antibody, a 20H6 antibody, a BH97 antibody, a 20H6 antibody, a 16CD11 antibody, a BH099 antibody, a BH18 antibody, and a BH30 antibody).

Nucleic acids encoding measles virus H polypeptides can be modified using common molecular cloning techniques (e.g., site-directed mutagenesis) to generate mutations at such positions. Possible mutations include, without limitation, substitutions (e.g., transitions and transversions), deletions, insertions, and combinations of substitutions, deletions, and insertions. Nucleic acid molecules can include a single nucleotide mutation or more than one mutation, or more than one type of mutation. Polymerase chain reaction (PCR) and nucleic acid hybridization techniques can be used to identify nucleic acids encoding measles virus H polypeptides having altered amino acid sequences.

Additional nucleic acid sequences can be included in a nucleic acid molecule provided herein. Such additional nucleic acid sequences include, without limitation, other viral sequences. For example, a nucleic acid molecule can contain a complete MV genomic sequence that includes, in a 5′-3′ direction, the N, P, M, F, H, and L sequences, wherein the naturally occurring H sequence is replaced by a sequence encoding a modified H polypeptide provided herein. A nucleic acid molecule containing such viral nucleic acid sequences can be used to transfect cells (e.g., CHO cells or 293.3.46 cells) in order to produce infectious virus particles. Alternatively, a nucleic acid molecule can contain sequences that encode a modified H polypeptide and an F polypeptide. Such a nucleic acid can contain an internal ribosome entry site (IRES) between the coding sequences.

A nucleic acid molecule provided herein can contain nucleic acid sequences such that the nucleic acid molecule encodes a replication-competent virus (e.g., replication-competent measles virus). For example, a nucleic acid molecule provided herein can contain viral sequences such that replication-competent viruses expressing modified H polypeptides are produced. As described herein, such a nucleic acid molecule can be an measles virus cDNA vector containing a nucleic acid sequence encoding a modified H polypeptide provided herein.

In some cases, a nucleic acid molecule provided herein can contain a nucleotide sequence such that the nucleic acid molecule encodes a replication-defective virus (e.g., replication-defective measles virus). For example, a nucleic acid molecule provided herein can contain viral sequences such that replication-defective viruses expressing or displaying modified H polypeptides are produced.

In some cases, a nucleic acid provided herein can encode a polypeptide that contains an H polypeptide amino acid sequence coupled to a second amino acid sequence. The second amino acid sequence can be from a polypeptide that is a ligand for a cell surface receptor or that binds to another polypeptide on a cell surface. For example, an amino acid sequence from a single chain antibody or from a growth factor can be used. The second amino acid sequence can be at the amino-terminal end of the amino acid sequence of the H polypeptide extracellular domain, or at the carboxy-terminal end of the H polypeptide amino acid sequence.

This document also provides vectors containing nucleic acid that encodes an H polypeptide provided herein. Such vectors can be, without limitation, viral vectors, plasmids, phage, and cosmids. For example, vectors can be of viral origin (e.g., paramyxovirus vectors, SV40 vectors, molecular conjugate vectors, or vectors derived from adenovirus, adeno-associated virus, herpes virus, lentivirus, retrovirus, parvovirus, or Sindbis virus) or of non-viral origin (e.g., vectors from bacteria or yeast). A nucleic acid encoding an H polypeptide provided herein can be inserted into a vector such that the H polypeptide is expressed. For example, a nucleic acid provided herein can be inserted into an expression vector. An expression vector can contain one or more expression control sequences (e.g., a sequence that controls and regulates the transcription and/or translation of another sequence). Expression control sequences include, without limitation, promoter sequences, transcriptional enhancer elements, and any other nucleic acid elements required for RNA polymerase binding, initiation, or termination of transcription.

A nucleic acid molecule provided herein can be obtained using any appropriate method including, without limitation, common molecular cloning and chemical nucleic acid synthesis techniques. For example, PCR can be used to construct nucleic acid molecules that encode a modified H polypeptide provided herein. PCR refers to a procedure or technique in which target nucleic acid is amplified in a manner similar to that described in U.S. Pat. No. 4,683,195, and subsequent modifications of the procedure described therein.

A nucleic acid provided herein can be incorporated into viruses by standard techniques. For example, recombinant techniques can be used to insert a nucleic acid molecule encoding a modified H polypeptide provided herein into an infective viral cDNA. In some cases, a nucleic acid can be exogenous to a viral particle, e.g., an expression vector contained within a cell such that the polypeptide encoded by the nucleic acid is expressed by the cell and then incorporated into a new viral particle.

### 2. Polypeptides

As used here, a “polypeptide” refers to a chain of amino acid residues, regardless of post-translational modification (e.g., phosphorylation or glycosylation). A measles virus H polypeptide provided herein (1) can be heterologous to naturally occurring H polypeptides or the H polypeptide having the amino acid sequence set forth in SEQ ID NO:2, and (2) can have reduced susceptibility to antibody neutralization as compared to a naturally occurring H polypeptide or an H polypeptide having the amino acid sequence set forth in SEQ ID NO:2 or SEQ ID NO:4.

A measles virus H polypeptide provided herein can contain one or more amino acid substitutions as compared to a naturally occurring measles virus H polypeptide or the measles virus H polypeptide having the amino acid sequence set forth in SEQ ID NO:2 or SEQ ID NO:4 such that measles viruses containing the encoded H polypeptide are less susceptible (e.g., two times, three times, four times, or more less susceptible) to antibody neutralization than measles viruses containing the corresponding, naturally occurring H polypeptide or measles viruses containing the measles virus H polypeptide having the amino acid sequence set forth in SEQ ID NO:2 or SEQ ID NO:4. For example, a measles virus H polypeptide provided herein can have the amino acid sequence set forth in SEQ ID NO:2 or SEQ ID NO:4 with the exception that the measles virus H polypeptide has an amino acid substitution at three or more (e.g., 3, 4, 5, 6, 7, 8, 9, 10, or more) of the following positions: P279, L284, Y310C, E395, K389, E398, K403, N405, D416, K488, E489, A490, E535, H536, A537, S546, R547, S550, F552, Y553, P554, S590, G592, E471, W472, P474, R547, and G316. In some cases, a measles virus H polypeptide provided herein can have the amino acid sequence set forth in SEQ ID NO:2 or SEQ ID NO:4 with the exception that the measles virus H polypeptide has three or more (e.g., 3, 4, 5, 6, 7, 8, 9, 10, or more) of the following amino acid substitutions: P279R, L284S, Y310C, E395K, K389R, E398G, K403N, N405T, D416N, K488N, E489A, A490T, E535N, H536A, A537T, S546G/R, R547A/C, S550T, F552N, Y553G, P554T, S590N, G592S, E471A, W472N, P474S, R547C, and G316N. Amino acid substitutions can be conservative or non-conservative. Conservative amino acid substitutions replace an amino acid with an amino acid of the same class, whereas non-conservative amino acid substitutions replace an amino acid with an amino acid of a different class. Examples of conservative substitutions include amino acid substitutions within the following groups: (1) glycine and alanine; (2) valine, isoleucine, and leucine; (3) aspartic acid and glutamic acid; (4) asparagine, glutamine, serine, and threonine; (5) lysine, histidine, and arginine; and (6) phenylalanine and tyrosine.

Non-conservative amino acid substitutions may replace an amino acid of one class with an amino acid of a different class. Non-conservative substitutions can make a substantial change in the charge or hydrophobicity of the gene product. Non-conservative amino acid substitutions also can make a substantial change in the bulk of the residue side chain, e.g., substituting an alanine residue for an isoleucine residue. Examples of non-conservative substitutions include the substitution of a basic amino acid for a non-polar amino acid or a polar amino acid for an acidic amino acid.

An H polypeptide amino acid sequence provided herein can be coupled to a second amino acid sequence. Such coupling can occur through, for example, peptide bonding. In some cases, a second amino acid sequence that can be coupled to an H polypeptide amino acid sequence provided herein can be that of a polypeptide having the ability to bind to cell surface receptors other than SLAM and CD46. In such cases, such second amino acid sequences can serve to target an H polypeptide provided herein to a particular type of cell (e.g., a tumor cell), depending on the receptor targeted by the second amino acid sequence. In some cases, a second amino acid sequence can be from a growth factor or a single chain antibody. A second amino acid sequence can be at the amino-terminal end of the amino acid sequence of the H polypeptide extracellular domain, or at the carboxy-terminal end of the H polypeptide amino acid sequence.

An H polypeptide that is incorporated into a virus can be encoded by a nucleic acid molecule that is present within the virus. Alternatively, a virus can take up an exogenous H polypeptide that is expressed by, for example, a cell.

H polypeptides can be produced using any appropriate method. For example, H polypeptides can be obtained by extraction from viruses, isolated cells, tissues and bodily fluids. H polypeptides also can be produced by chemical synthesis. In some cases, H polypeptides provided herein can be produced by standard recombinant technology using heterologous expression vectors encoding H polypeptides. Expression vectors can be introduced into host cells (e.g., by transformation or transfection) for expression of the encoded polypeptide, which then can be purified. Expression systems that can be used for small or large scale production of H polypeptides include, without limitation, microorganisms such as bacteria (e.g., E. coli and B. subtilis) transformed with recombinant bacteriophage DNA, plasmid DNA, or cosmid DNA expression vectors containing a nucleic acid molecule provided herein, and yeast (e.g., S. cerevisiae) transformed with recombinant yeast expression vectors containing a nucleic acid molecule provided herein. Useful expression systems also include insect cell systems infected with recombinant virus expression vectors (e.g., baculovirus) containing a nucleic acid molecule provided herein, and plant cell systems infected with recombinant virus expression vectors (e.g., tobacco mosaic virus) or transformed with recombinant plasmid expression vectors (e.g., Ti plasmid) containing a nucleic acid molecule provided herein. H polypeptides also can be produced using mammalian expression systems, which include cells (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with a nucleic acid molecule provided herein.

### 3. Viruses

This document also provides viruses containing a nucleic acid molecule and/or an H polypeptide provided herein. For example, this document provides recombinant viruses that contain a measles virus H polypeptide that (1) is heterologous to naturally occurring H polypeptides or the H polypeptide having the amino acid sequence set forth in SEQ ID NO:2, and (2) has reduced susceptibility to antibody neutralization as compared to a naturally occurring H polypeptide or the H polypeptide having the amino acid sequence set forth in SEQ ID NO:2.

Viruses containing a nucleic acid molecule provided herein are not required to express the encoded measles virus H polypeptide. For example, a virus (e.g., an adenovirus) can be engineered to contain a nucleic acid that encodes an H polypeptide provided herein. In this case, the engineered virus may or may not express the encoded H polypeptide. Viruses containing nucleic acid that encodes an H polypeptide can be used to deliver the nucleic acid to cells, such that the cells express the encoded H polypeptide.

In some cases, viruses that contain a nucleic acid molecule described herein can express the encoded H polypeptide. For example, a measles virus containing a nucleic acid molecule that encodes a modified H polypeptide can display the modified H polypeptide on its surface. Such a virus can target cells for viral entry while having a reduced susceptibility to antibody neutralization.

Any appropriate virus can contain a nucleic acid molecule encoding a modified measles virus H polypeptide provided herein. Viruses can be RNA viruses or DNA viruses. Viruses can be, for example, nonsegmented negative strand RNA viruses belonging to the Mononegavirales group (e.g., measles virus, human parainfluenzavirus, rabies virus, respiratory syncytial virus, and mumps virus). Viruses also can be influenza viruses, which have a segmented RNA genome of negative polarity and share several structural features with measles virus. Viruses also can be, without limitation, enveloped viruses such as herpes simplex virus, and retroviruses such as murine leukemia virus and human immunodeficiency virus.

A virus provided herein can be attenuated. As used herein, the term “attenuated” refers to a virus that is immunologically related to a wild type virus but which is not itself pathogenic. An attenuated measles virus, for example, does not produce classical measles disease. Attenuated viruses typically are replication-competent, in that they are capable of infecting and replicating in a host cell without additional viral functions supplied by, for example, a helper virus or a plasmid expression construct encoding such additional functions.

Any appropriate method can be used to identify a virus containing a nucleic acid molecule provided herein. Such methods include, without limitation, PCR and nucleic acid hybridization techniques such as Northern and Southern analysis. In some cases, immunohistochemistry and biochemical techniques can be used to determine if a virus contains a particular nucleic acid molecule by detecting the expression of a polypeptide encoded by that particular nucleic acid molecule.

### 4. Methods of Using H Polypeptides, Nucleic Acids, and Viruses

H polypeptides and/or nucleic acids provided herein can be administered to cells in order to induce cell fusion. For example, a nucleic acid molecule (e.g., a viral vector) encoding a modified H polypeptide as well as any other polypeptide (e.g., an F polypeptide) can be administered to a tumor in order to induce fusion between tumor cells, ultimately resulting in cell death.

Viruses that contain nucleic acids and/or H polypeptides provided herein also can be administered to cells (e.g., in vivo or in vitro) to induce cell fusion. Incorporation of the encoded H polypeptide into the virus is not required. For example, a virus (e.g., an adenovirus) can be engineered to contain a nucleic acid encoding an H polypeptide that is not incorporated into the virus. Such a virus can be administered to a cell population in order to deliver the nucleic acid encoding the H polypeptide into the cells. The infected cells then can express the encoded H polypeptide, leading to cell fusion. Viruses provided herein that contain nucleic acids encoding H polypeptides and contain the H polypeptides also are useful for inducing cell fusion. In some cases, such a virus can target cells while having a reduced susceptibility to antibody neutralization.

A measles virus, for example, that contains a nucleic acid encoding an H polypeptide also can contain the encoded H polypeptide. Such a virus can be used to target particular cells as described herein while having a reduced susceptibility to antibody neutralization. The H polypeptide then can be expressed within the targeted cells, inducing the cells to fuse. It is noted that an F polypeptide can be used with the H polypeptides provided herein. For example, a single virus can contain nucleic acid that encodes both an H polypeptide and an F polypeptide.

Viruses provided herein can be used to treat cancer patients. A particular virus can be propagated in host cells in order to increase the available number of copies of that virus, typically by at least 2-fold (e.g., by 5- to 10-fold, by 50- to 100-fold, by 500- to 1,000-fold, or even by as much as 5,000- to 10,000-fold). A virus can be expanded until a desired concentration is obtained in standard cell culture media (e.g., DMEM or RPMI-1640 supplemented with 5-10% fetal bovine serum at 37° C. in 5% CO₂). A viral titer typically is assayed by inoculating cells (e.g., Vero cells) in culture. After 2 to 3 hours of viral adsorption, the inoculum is removed, and cells are overlaid with a mixture of cell culture medium and agarose or methylcellulose (e.g., 2 ml DMEM containing 5% FCS and 1% SeaPlaque agarose). After about 3 to about 5 days, cultures are fixed with 1 mL of 10% trifluoroacetic acid for about 1 hour, then UV cross-linked for 30 minutes. After removal of the agarose overlay, cell monolayers are stained with crystal violet and plaques are counted to determine viral titer. Virus is harvested from infected cells by scraping cells from the dishes, subjecting them to freeze/thawing (e.g., approximately two rounds), and centrifuging. The cleared supernatants represent “plaque purified” virus.

Viral stocks can be produced by infection of cell monolayers (e.g., adsorption for about 1.5 hours at 37° C.), followed by scraping of infected cells into a suitable medium (e.g., Opti-MEM; Gibco/Invitrogen, Carlsbad, Calif.) and freeze/thaw lysis. Viral stocks can be aliquoted and frozen, and can be stored at −70° C. to −80° C. at concentrations higher than the therapeutically effective dose. A viral stock can be stored in a stabilizing solution. Stabilizing solutions are known in the art and include, without limitation, sugars (e.g., trehalose, dextrose, glucose), amino acids, glycerol, gelatin, monosodium glutamate, Ca²⁺, and Mg²⁺.

The methods and materials provided herein can be used to treat cancer (e.g., to reduce tumor size, inhibit tumor growth, or reduce the number of viable tumor cells). As used herein, “reducing the number of viable tumor cells” is meant to encompass (1) slowing the rate of growth of a population of tumor cells such that after a certain period of time, a tumor in a treated individual is smaller than it would have been without treatment; (2) inhibiting the growth of a population of tumor cells completely, such that a tumor stops growing altogether after treatment; and/or (3) reducing the population of tumor cells such that a tumor becomes smaller or even disappears after treatment.

Viruses provided herein can be administered to a cancer patient by, for example, direct injection into a group of cancer cells (e.g., a tumor) or intravenous delivery to cancer cells. Types of cancer cells susceptible to treatment with viruses include neuronal cells, glial cells, myelomonocytic cells, and the like. The methods provided herein can be used to treat types of cancer that include, but are not limited to, myeloma, melanoma, glioma, lymphoma, and cancers of the lung, brain, stomach, colon, rectum, kidney, prostate, ovary, and breast. An attenuated measles virus containing a modified H polypeptide provided herein can be used to treat, for example, a lymphoma (e.g., non-Hodgkin's Lymphoma).

Viruses containing polypeptides that have H polypeptide amino acid sequences coupled to a second amino acid sequence can be particularly useful for treating cancer or reducing tumor growth. For example, a virus containing an H polypeptide coupled to a growth factor amino acid sequence can target cells (e.g., tumor cells) that have growth factor receptors on their surface.

A virus provided herein can be administered to a patient in a biologically compatible solution or a pharmaceutically acceptable delivery vehicle, by administration either directly into a group of cancer cells (e.g., intratumorally) or systemically (e.g., intravenously). Suitable pharmaceutical formulations depend in part upon the use and the route of entry, e.g., transdermal or by injection. Such forms should not prevent the composition or formulation from reaching a target cell (i.e., a cell to which the virus is desired to be delivered to) or from exerting its effect. For example, pharmacological compositions injected into the blood stream should be soluble.

While dosages administered will vary from patient to patient (e.g., depending upon the size of a tumor), an effective dose can be determined by setting as a lower limit the concentration of virus proven to be safe as a vaccine (e.g., 10³ pfu) and escalating to higher doses of up to 10¹² pfu, while monitoring for a reduction in cancer cell growth along with the presence of any deleterious side effects. A therapeutically effective dose typically provides at least a 10% reduction in the number of cancer cells or in tumor size. Escalating dose studies can be used to obtain a desired effect for a given viral treatment (see, e.g., Nies and Spielberg, “Principles of Therapeutics,” In Goodman & Gilman's The Pharmacological Basis of Therapeutics, eds. Hardman, et al., McGraw-Hill, N.Y., 1996, pp 43-62).

Viruses provided herein can be delivered in a dose ranging from, for example, about 10³ pfu to about 10¹² pfu (typically >10⁸ pfu). A therapeutically effective dose can be provided in repeated doses. Repeat dosing is appropriate in cases in which observations of clinical symptoms or tumor size or monitoring assays indicate either that a group of cancer cells or tumor has stopped shrinking or that the degree of viral activity is declining while the tumor is still present. Repeat doses (using the same or a different modified virus) can be administered by the same route as initially used or by another route. A therapeutically effective dose can be delivered in several discrete doses (e.g., days or weeks apart). In some cases, one to about twelve doses can be provided. In some cases, a therapeutically effective dose of attenuated measles virus can be delivered by a sustained release formulation.

Viruses provided herein can be administered using a device for providing sustained release. A formulation for sustained release of a virus can include, for example, a polymeric excipient (e.g., a swellable or non-swellable gel, or collagen). A therapeutically effective dose of a virus can be provided within a polymeric excipient, wherein the excipient/virus composition is implanted at a site of cancer cells (e.g., in proximity to or within a tumor). The action of body fluids gradually dissolves the excipient and continuously releases the effective dose of virus over a period of time. In some cases, a sustained release device can contain a series of alternating active and spacer layers. Each active layer of such a device typically contains a dose of virus embedded in excipient, while each spacer layer contains only excipient or low concentrations of virus (i.e., lower than the effective dose). As each successive layer of the device dissolves, pulsed doses of virus are delivered. The size/formulation of the spacer layers determines the time interval between doses and is optimized according to the therapeutic regimen being used.

A virus provided herein can be directly administered. For example, a virus can be injected directly into a tumor (e.g., a lymphoma) that is palpable through the skin. Ultrasound guidance also can be used in such a method. In some cases, direct administration of a virus can be achieved via a catheter line or other medical access device, and can be used in conjunction with an imaging system to localize a group of cancer cells. By this method, an implantable dosing device typically is placed in proximity to a group of cancer cells using a guidewire inserted into the medical access device. An effective dose of a virus also can be directly administered to a group of cancer cells that is visible in an exposed surgical field.

In some cases, viruses provided herein can be delivered systemically. For example, systemic delivery can be achieved intravenously via injection or via an intravenous delivery device designed for administration of multiple doses of a medicament. Such devices include, but are not limited to, winged infusion needles, peripheral intravenous catheters, midline catheters, peripherally inserted central catheters, and surgically placed catheters or ports.

The course of virus therapy can be monitored by evaluating changes in clinical symptoms (known in the art for each particular type of cancer) or by direct monitoring of the size of a group of cancer cells or tumor. A method for using a virus of the invention to treat cancer is considered effective if the cancer cell number, tumor size, tumor specific antigen level, and/or other clinical symptoms are reduced by at least 10 percent following administration of virus. For a solid tumor, for example, the effectiveness of virus treatment can be assessed by measuring the size or weight of the tumor before and after treatment. Tumor size can be measured either directly (e.g., using calipers), or by using imaging techniques (e.g., X-ray, magnetic resonance imaging, or computerized tomography) or from the assessment of non-imaging optical data (e.g., spectral data). For a group of cancer cells (e.g., leukemia cells), the effectiveness of viral treatment can be determined by measuring the absolute number of leukemia cells in the circulation of a patient before and after treatment. The effectiveness of viral treatment also can be assessed by monitoring the levels of a cancer specific antigen. Cancer specific antigens include, for example, carcinoembryonic antigen (CEA), prostate specific antigen (PSA), prostatic acid phosphatase (PAP), CA 125, alpha-fetoprotein (AFP), carbohydrate antigen 15-3, and carbohydrate antigen 19-4.

The measles viruses provided herein can be used to vaccinate humans (e.g., infants less than 9 months of age or infants less than 15 months of age). When vaccinating an infant less than 9 months or 15 months of age using the measles viruses provided herein as a vaccine, the vaccine can effectively induce a protective immune response against measles virus infection even though the infant contains maternal anti-measles virus antibodies.

The invention will be further described in the following examples, which do not limit the scope of the invention described in the claims.

### EXAMPLES

### Example 1

### Combing Rational Design and Natural Selection to Engineer a Measles Virus Resistant to a Cocktail of Monoclonal Antibodies After Modifying the H Polypeptide with Glycan Shields and Escape Mutations

### Cell Culture

Vero African green monkey kidney cells and Chinese hamster ovary (CHO) cells were grown in DMEM with 5% FBS. CHO-CD46 cells (Nakamura et al., Nat. Biotechnol., 22:331-336 (2004)) stably expressing human CD46 were maintained in DMEM, 10% FBS, 1 mg of G418 per mL. CHO-SLAM cells (Tatsuo et al., Nature, 406:893-897 (2000)) stably expressing human SLAM were grown in RPMI 1640, 10% FBS, 0.5 mg/mL of G418. CHO-Nectin-4 cells (obtained from Marc Lopez) stably expressing human Nectin-4 were maintained in RPMI 1640, 10% FCS, 0.5 mg/mL of G418, 0.1 mM MEM non-essential amino acids solution. All cell culture media were supplemented with 1% penicillin and streptomycin, and cells were grown at 37° C. in a humidified atmosphere of 5% CO₂.

### Site-Directed Mutagenesis of Rationally Designed Mutations

Mutations in H were performed in pCG-H (measles H glycoprotein (Nse strain) expression construct) using QuikChange® Site-Directed Mutagenesis Kit (Stratagene) as per manufacture's directions (Publication Number: 200518-12). Overlapping oligonucleotides were designed to introduce mutations in the H gene using pfuTurbo DNA polymerase (Stratagene). Following DpnI digestion of parental plasmid DNA, the synthetic DNAs were transfected into DH5a competent cells (Invitrogen). DNA sequence analysis was used to confirm the intended mutations and to ensure that the plasmids were free of unwanted second-site mutations. Each H mutant was designated a number (#), i.e. pCG-H5, H14, H11, H22.

### Measles Fusion Assay

After the mutant H genes were confirmed by sequencing, the mutations were tested for bioactivity using a cell fusion assay. 6-well plates were seeded with 4 mL of Vero cells (3×10⁶) in Dulbecco's Modified Eagle's Medium (DMEM, Lonza) supplemented with 5% Fetal Bovine Serum (FBS, Lonza) 24 hours in advance of the assay. The plates were incubated at 37° C. in a humidified environment containing 5% CO₂. The next day, the media was removed from the cells and replaced with 1 mL fresh, pre-warmed DMEM with 0% FBS. 2 μg of a plasmid encoding the measles fusion gene (F) was mixed with 2 μg of WT or mutant H DNA in a polystyrene tube, and pre-warmed DMEM-0% was added to a total volume of 50 μL. 7 μL of FuGene (Roche) was directly added to the DNA mixture, and the tubes were vortexed and incubated at room temperature for 15 minutes. The DNA-FuGene mixture was added to each well drop-wise and mixed by gently rocking the plate side to side. The plate was incubated at 37° C. for 30 minutes, and then rocked again and incubated at 37° C. for 5 hours. 2 mL of DMEM- 5% was added to each well, rocked to mix, and returned to 37° C. to incubate. Plates were examined under a phase-contrast microscope to observe cell fusion over the next three days. Fused foci of cells (syncytia) were scored on a three point scale (+, +/−, and −).

### Plasmids, Virus Rescue, and Titration

To generate the full-length anti-genomic MV plasmids, in which the H glycoprotein was replaced by H#, p+MVeGFP (Duprex et al., J. Virol., 73:9568-9575 (1999)) and the pCG-H# expression constructs were digested with PacI and SpeI, and H(Nse strain) was exchanged with the H# fragments to create the plasmids p+MVeGFP-H# (p+MV-H#). Correct assembly of the constructs was verified by dideoxy-sequencing. MV rescue system was used as described elsewhere (Radecke et al., EMBO J., 14:5773-5784 (1995)). Briefly, 293-3-46 cells were transfected with p+MVeGFP-H# or p+MVeGFP and pEMCLa plasmid using a calcium phosphate transfection method. Seventeen hours post transfection, media was replaced with fresh media, and the cells were heat-shocked at 42° C. for 2.5 hours. Seventy-two hours after transfection, the 293-3-46 cells were over-layed onto Vero cells. Twenty-four hours later, eGFP expressing syncytia started appearing. Individual syncytia (clones) were picked and used to infect new Vero cells to amplify the virus. The virus was further amplified with two serial passages by infecting Vero cells at moi of 0.02 at 37° C. When 90% of the cells were incorporated into syncytia, the cells were harvested by scrapping them off the plate in 1-2 mL of Opti-MEM (Invitrogen). The cell/virus suspension was subjected to three freeze-thaw cycles, centrifugated to clarify viral supernatant, aliquoted, and stored at −80° C. Titers were determined by 50% tissue culture infective dose (TCID₅₀) on Vero cells. Titer in PFU=0.7*titer in TCID₅₀.

### Establishing a Neutralizing Concentration of mAb

The ability of mAbs to inhibit MV-eGFP infection was assayed by the plaque reduction neutralization test (PRNT) in a 96 well format. mAbs were serially diluted in Opti-MEM (Invitrogen), and 50 μL was mixed with equal volume of MV-eGFP at 400 PFU/mL. The mixture was incubated for 1.5 hours at 37° C. before the addition of Vero cells (10,000 cells/well in 50 μL DMEM 5% FBS). Forty-eight hours post infection, the number of eGFP expressing syncytia/well were counted using a fluorescent microscope. The neutralizing concentration of mAb was the endpoint concentration of mAb required to reduce the number of MV-eGFP syncytia by 100%. Neutralization concentrations established for each mAb were as follows. 16DE6 (1.5 μL ascites/well), I-41 (1 μL ascites/well), I-44 (1.5 μL ascites/well), I-29 (6 μL ascites/well), BH141 (5 μL ascites/well), BH97 (2.5 μg/well), BH15 (3-6 μg/well) BH30 (0.8 μg/well), c148 (1 μg/well), and c87 (50 μL hybridoma supernatant).

### Neutralization Assay

Neutralization assay was performed in a 96 well format. Each sample was duplicated, and the experiment was repeated 2 to 5 times depending on the mAb. Neutralizing concentration of mAb in 50 μL Opti-MEM or Opti-MEM alone (control) was mixed with equal volume of MV-eGFP or MV-H mutant viruses (400 PFU/mL). In the case of mAb c87, 50 μL of hybridoma supernatant or 50 μL media were mixed with equal volume of virus. The mixture was incubated for 1.5 hours at 37° C. before the addition of Vero cells (10,000 cells/well in 50 μL DMEM 5% FBS). Forty-eight hours post infection, the number of eGFP expressing syncytia/well were counted using a fluorescent microscope.

### Natural Selection

Vero Cells were seeded into six-well plates at a density of 2.5×10⁵ cells/well and left to adhere overnight. Prior to infection, MV-H11 (1×10⁶PFU) was incubated at 37° C. for 2 hours in with 300 μg/mL c148. MV-δE3 was incubated with 40 μg/mL BH38. The virus/antibody mixture was split between 6 wells of Vero cells. Viral replication was monitored for 72-96 hours. Virus from wells that exhibited the greatest degree of propagation was harvested and challenged again with antibody. This was repeated twice before syncytia were picked and amplified in 6 well plates in the presence of 40 μg c148 or 10 μg BH38/well. Viral H genes were amplified by polymerase chain reaction (PCR) from individual clones and analyzed by dideoxy-sequencing to identify the escape mutation. Escape mutant propagation and titrations was performed as described herein.

### Structural Modeling

The crystal structure of measles virus hemagglutinin protein PDB ID: 2ZB6 were analyzed and manipulated using PyMOL software.

### Monoclonal Antibodies

Monoclonal antibodies used in this study were obtained from Drs. Claude Muller (Laboratoire National de Sante, Luxemburg; BH15, BH97, BH30, BH38, BH15), Erling Norrby and Mariethe Ehnlund (Karolinska Institute, Sweden; 16DE6, I-41, I-44, I-29), Denis Gerlier (Inserm, France; c87), and Mark Federspiel (Mayo Clinic, USA; purified c148), but originally from T. Fabian Wild.

### Infection of CHO Cells

CHO, CHO-CD46, CHO-Nectin-4, and CHO-SLAM cells were plated in a 96 well format and infected in triplicate. On the day of infection, media were removed from the cells, and 50 μL of virus were added in Opti-MEM (Invitrogen). Cells were infected with recombinant measles viruses at a moi of 1 (moi calculated based on TCID₅₀/mL as titered on Vero cells). Two hours post infection, 100 μL of media (cell type dependent) were added to the cells. Seventeen hours later, the media were replaced with fresh media or media with 200 μM fusion inhibitory peptide (FIP) (Bachem). Fluorescence microscopy was performed 48 and 72 hours post infection.

### Results

The control virus for these experiments was MV-eGFP. This is a highly attenuated, laboratory adapted strain of vaccine Edmonston B ancestry expressing eGFP (Duprex et al., J. Virol., 73:9568-9575 (1999)). The H gene within MV-eGFP is a Edmonston-tag strain. MV-eGFP propagates in Vero cells using the CD46 receptor, but still has tropism for human SLAM and Nectin-4. All engineered and naturally selected mutations presented herein were within the H gene encoded by MV-eGFP, and the mutated viruses were renamed as indicated.

### Measles Virus H can Tolerate Rather Significant Escape Mutations Including N-Linked Glycosylation Sites in Different Immunodominant Epitopes

The rational design approach used the technology of immune dampening (Nara et al., PLoS Biol., 8:e1000571 (2010); and Tobin et al., Vaccine, 26:6189-6199 (2008)) to shield potential immunodominant epitopes from neutralizing monoclonal antibodies (n-mAbs). Epitopes were modified with rationally designed potential N-linked glycosylation sites (PNGS) (Table 1) and other amino acids substitutions. The rational design of potential escape mutations relied strongly on the analysis of the H crystal structure and previously published immunodominant epitopes and monoclonal antibody (mAb) escape mutations (Bouche et al., Viral Immunol., 15:451-471 (2002); Santiago et al., Nat. Struct. Mol. Biol., 17:124-129 (2010); Hashiguchi et al., Nat. Struct. Mol. Biol., 18:135-141 (2011); Hashiguchi et al., Proc. Natl. Acad. Sci. USA, 104:19535-19540 (2007); Hu et al., Virology, 192:351-354 (1993); Giraudon and Wild, Virology, 144:46-58 (1985); Ertl et al., Arch. Virol., 148:2195-2206 (2003); and de Carvalho Nicacio et al., J. Virol., 76:251-258 (2002). To permit viral replication, rationally-designed mutations within epitopes were generally placed in flexible surface loops, and residues that interacted with receptors were avoided.

TABLE 1
Loops identified as potential antigenic sites.

| Potential immunodominant loops | Examples of PNGS | mAb escape |
|---|---|---|
| H236-250 | | |
| H261-269 | R261K | |
| H280-285 | 282NDL -> NDS | BH15 |
| H307-318 | | I-29, BH141, |
| | | BH30 |
| H403-407 | 403KDN -> NDT | Cl48, I-44 |
| H445-451 | N450Y | |
| H471-477 | 472WIP -> NIS | BH30 |
| H487-495 | 488KEA -> NAT | 16CD11 |
| H530-537 | 535EHA -> NAT | 16DE6, c87, |
| | | cl18, c8, I-41 |
| H552-562 | 552FYP -> NGT | C87, cl18, I-41 |
| H586-595 | 590SGG -> NGS | |

PNGS, potential N-linked glycosylation site. Defined by a sequon triplet of NXS/T, where X is any amino acid except for P. Potential N-linked glycosylation sites (PNGS) can be inserted or mutated into any position within the defined loop to cloak the epitopes from antibody recognition.

The H protein was subjected to rounds of mutagenesis, while being encoded in an expression plasmid. Following mutagenesis, the bioactivity of H was monitored in vitro as the ability to induce cell-cell fusion of Vero cells when co-expressed with MV fusion (F) protein (Table 2). Bioactive H mutants, encoding mutations in 1-2 potential epitopes, were cloned into MV-eGFP, and a panel of viruses with mutated H proteins (MV-H#) was rescued. All MV-H# viruses grew to comparable titers as the control virus MV-eGFP, reaching 10⁷ TCID₅₀/mL following three passages (Table 2).

TABLE 2
MV-H# viruses with mutations in potential immunodominant
epitopes screened to identify monoclonal escape mutations.

| | | | | | | |
|---|---|---|---|---|---|---|
| | Ratioanlly designed | | TCID₅₀/ | R. human | | |
| MV-H# | mutations | Syncitia | mL | serum * | R. to mAb | Eptiopes |

Amino Acid Substitutions

| | | | | | | |
|---|---|---|---|---|---|---|
| H3 | E395D, N396Q, E398D | yes | 10⁷ | no | Cl48, I-44 | E3 |
| H4 | Q391G, E395A, N396G, | yes | 10⁷ | no | Cl48, I-44 | E3 |
| | E398G, | | | | | |
| | A400V | | | | | |
| H5 | Q383N, A385G, K387R, | yes | 10⁷ | no | Cl48, I-44 | E3 |
| | G388A, E395D, | | | | | |
| | N396G, E398D | | | | | |
| H7 | R547G | Yes | 10⁷ | No | Neutralized by mAb | |
| | | | | | screened | |
| H8 | S546G, R547A, S550T | Yes | 10⁷ | No | c87, BH97, I-41 | E2, E5 |

Engineered Potential N-linked glycosylation sites

| | | | | | | |
|---|---|---|---|---|---|---|
| H14 | 282 NDL −> NDS | Yes | 10⁷ | No | BH15 | E1 |
| H23 | 282 NDL −> NDS, E398G | Yes | 10⁷ | No | BH15 | E1 |
| H11 | 282 NDL −> NDS, | Yes | 10⁷ | no | BH15, 16DE6, c87, | E1, E2 |
| | 535 EHA −>NAT, | | | | | |
| | E398G, | | | | cl18, c8, I-41 | |
| H16 | 403 KDN −> NDT | Yes | 10⁷ | No | cl48, I-44 | E3 |
| H21 | 551 FYP −> NGT | Yes | 10⁷ | No | c87, cl18, c8, I-41 | E2 |
| H20 | 590 SGG −> NGS | Yes | 10⁷ | No | Neutralized by mAb | |
| | | | | | screened | |
| H22 | 590 SGG −> NGS, Y310T | Yes | 10⁷ | No | I-29, BH141 | E4 |
| H27 | 590 SGG −> NGS, | Yes | | ND | ND | ND |
| | 488 KEA −>NAT, Y310T | | | | | |

Control

| | | | | |
|---|---|---|---|---|
| MVeGFP | yes | 10⁷ | No | Neutralized by mAb |
| | | | | screened |

* purchased human AB serum, sterile filtered, heat inactivated;
ND, not done;
TCID₅₀, 50% Tissue Culture Infectious Dose

Since only one to two potential epitopes were modified within H per MV-H# virus, it was not expected that these modifications would confer resistance to pooled human serum. As expected, all viruses subjected to the screen were equally sensitive to pooled human serum as control MV-eGFP in a plaque reduction neutralization assay (PRNT) (Table 2).

### Screen of MV-H Mutants Identifies Four Non-Overlapping Epitopes Surrounding the Top Half of the H Cuboidal Head Domain

To better delineate the epitopes to shield from n-mAbs and determine the degree of protection the engineered mutations offered, the panel of MV-H# viruses were screened against a panel of n-mAbs in an in vitro neutralization assay (Table 2). A plaque reduction neutralization test (PRNT) was initially performed to determine the neutralizing concentration of each mAb (concentration required to neutralize MV-eGFP by 100%). A neutralizing concentration of n-mAb was then used for all subsequent neutralization assays to screen MV-H mutants. The neutralizing assay was performed in a 96 well format. Twenty to thirty PFU of MV-H# per well were incubated in neutralizing concentrations of mAb or media (control) prior to infection. Two days following infection, the number of infectious foci were quantified by counting the number of eGFP expressing syncytia per well. A virus escaped neutralization if the number of syncytia in the presence of mAb was the same as in the absence of mAb (control). Partial resistance was considered following up to a 40% reduction in infection in the presence of mAb compared to a 100% reduction of a control virus.

Escape mutations identified in this screen were used to cluster n-mAbs into four groups targeting four non-overlapping epitopes: E1-E4 (Table 3). These included n-mAbs thought to target the receptor binding interface, I-29, I-41, 16DE6, and I-44. These mAb had previously been shown to inhibit binding between soluble H protein and receptors CD46 and SLAM in in vitro binding studies (Santiago et al., J. Biol. Chem., 277:32294-32301 (2002)).

TABLE 3
Mutations in MV-H# escape mutants delineate four different epitopes targeted by mAb.

| MV-H# | Rationally designed mutations | Escape mutations | mAb escape | Designated Epitope |
|---|---|---|---|---|
| MV-eGFP | none | — | — | — |
| MV-H14 | 282 NDL -> NDS | 282 NDL -> NDS | BH15 | E1 |
| MV-H11 | 282 NDL -> NDS, 535 | 282 NDL -> NDS | BH15 | E1 |
| | EHA -> NAT, E398G | 535 EHA -> NAT | 16DE6, I-41, c87 | E2 |
| MV-H5 | Q383N, A385G, K387R, G388A, | Not determined | I-44, cl48 | E3 |
| | E395D, N396G, E398D | | | |
| MV-H22 | Y310T, 590 SGG -> NGS | Y310T | BH141, I-29 | E4 |
| — | Previously published escape | Y310D, L296I | BH38 | E4 |
| | mutants | | | |

Protection against antibodies recognizing these epitopes can be combined using rational design and natural selection to shield multiple epitopes from a polyclonal Ab response.

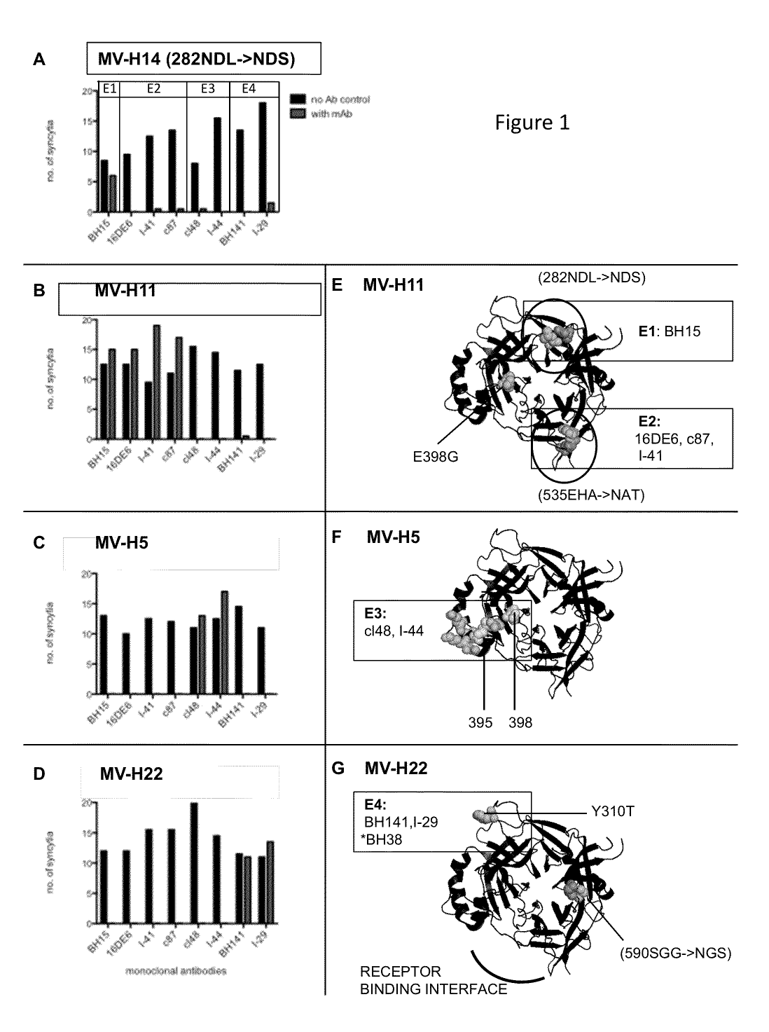

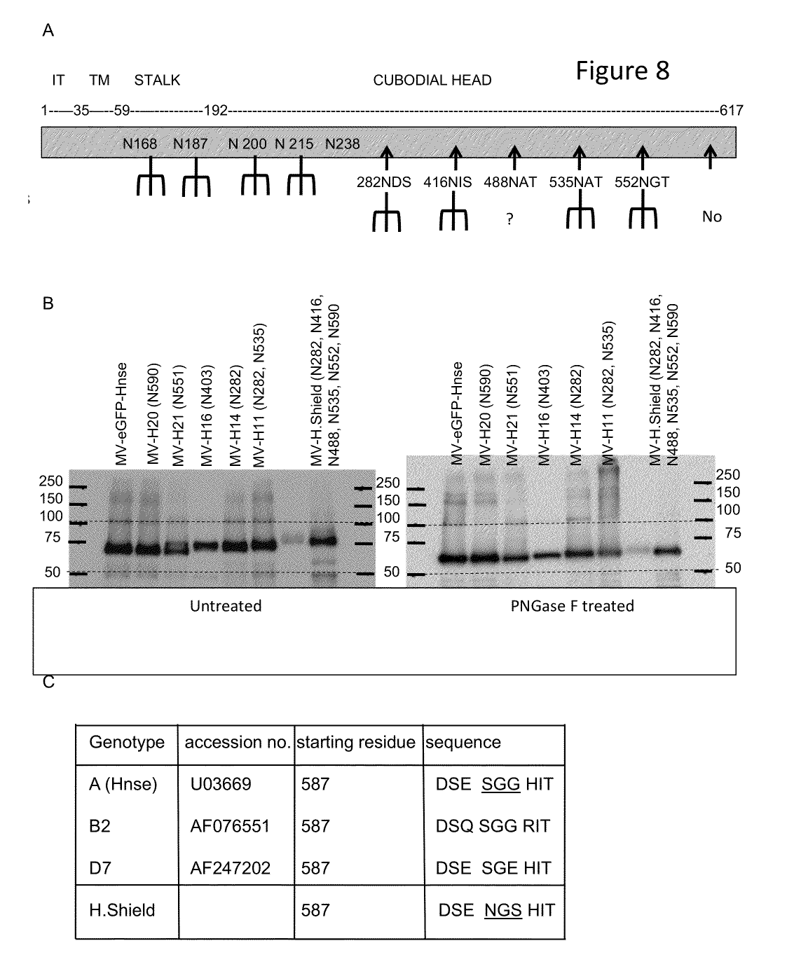

E1-4 were mapped onto the crystal structure of the H cuboidal head ectodomain (2ZB6.PDB) (FIG. 1) based on the location of the escape mutations identified in the screen (Table 2 and Table 3). E1-4 mapped to the top half of the head domain (FIG. 1 E-G) so only the top view of the head domain is shown in the structural figures. E3 was protected by mutations in MV-H3-H5 and H16 (Table 2). Since all these mutations clustered together, only MV-H5 were mapped to illustrate their general location on the structure (FIG. 1F). None of the rationally designed MV-H# mutants screened escaped BH38, but previous studies identified BH38 escape mutations as Y310D and L296I (Bouche et al., Viral Immunol., 15:451-471 (2002)). Since, residue 310 formed part of the E4 epitope, BH38 was grouped into E4 (FIG. 1D and FIG. 1G). N-linked glycosylation of rationally designed PNGS, protecting El (282NDL→NDS) and E2 (535EHA→NAT), was confirmed by western blotting of untreated and PNGaseF treated viral H proteins (FIG. 8). The PNGS at 590 SGG→NGS (FIG. 1D and FIG. 1G) was not glycosylated (FIG. 8). This mutation also was not responsible for mAb resistance (MV-H20, Table 2). Hence, only residue 310 forms part of the E4 epitope in MV-H22.

### Generation of a MV-H Mutant Escaping Monoclonal Antibodies Targeting Three Different Epitopes (MV-δE3)

An aim was to generate a MV-H mutant with minimal escape mutations protecting E1-E4 from neutralizing monoclonal antibodies (n-mAbs). MV-H11 already had two N-linked glycan shields at N282 and N535 shielding E1 and E2 (FIG. 1E). To add escape mutations that would protect E3 from n-mAbs, MV-H11 was naturally selected in neutralizing concentrations of mAb c148 recognizing E3. The rational behind the natural selection protocol was based on the fact that RNA viruses existed as a quasispecies population, due to the low fidelity of the RNA polymerase. MV mutation rate is of the order of ˜10⁻⁵ per nucleotide per replication cycle, which equates to about one mutation per genome.

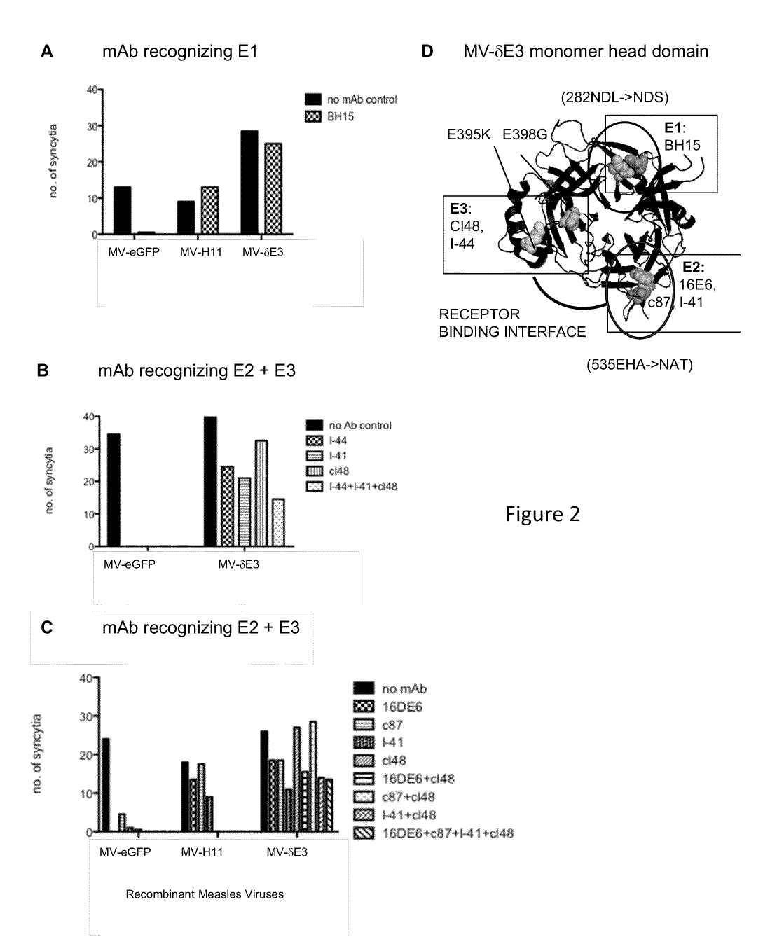

Following natural selection of MV-H11 in c148, a resistant clone with the escape mutation E395K was isolated, and the virus was called MV-δE3 (FIG. 2D). The initial screen showed that MV-H3-H5 (Table 2) completely or partially escaped c148 and I-44. These viruses all had a mutation in residue 395 as well as 398. A single E398G mutation in MV-H 11 and MV-H23 (Table 2) did not protect E3, but may be complimentary in the context of E395K in MV-δE3.

MV-δE3 was either partially or completely resistant to n-mAb targeting E1-3 independently or in different combinations (FIGS. 2A-C). n-mAbs targeting E2-3 were mixed in different combinations, as these epitopes were on the outskirts of the receptor-binding interface. Neutralizing mAb I-41 reduced MV-δE3 infection by 50%, indicating that additional protective mutations are required in E2 to provide complete protection from I-41, in the context of mutations present in MV-δE3. BH97 targets a fifth epitope, E5 (Table 2). BH97 was used as a positive control for MV neutralization. It also allowed for monitoring if the accumulation of escape mutations in different epitopes inadvertently provided protection against BH97, as this would imply a structural change in the head domain following selection. Natural selection did not disrupt pre-existing resistance to monoclonal antibodies targeting E1 and E2 (FIGS. 2A-C) and so this technique was continued to select escape mutations in E4.

### Generation of a MV-H Mutant Escaping Monoclonal Antibodies Targeting Four Different Epitopes (MV-δ4) Simultaneously

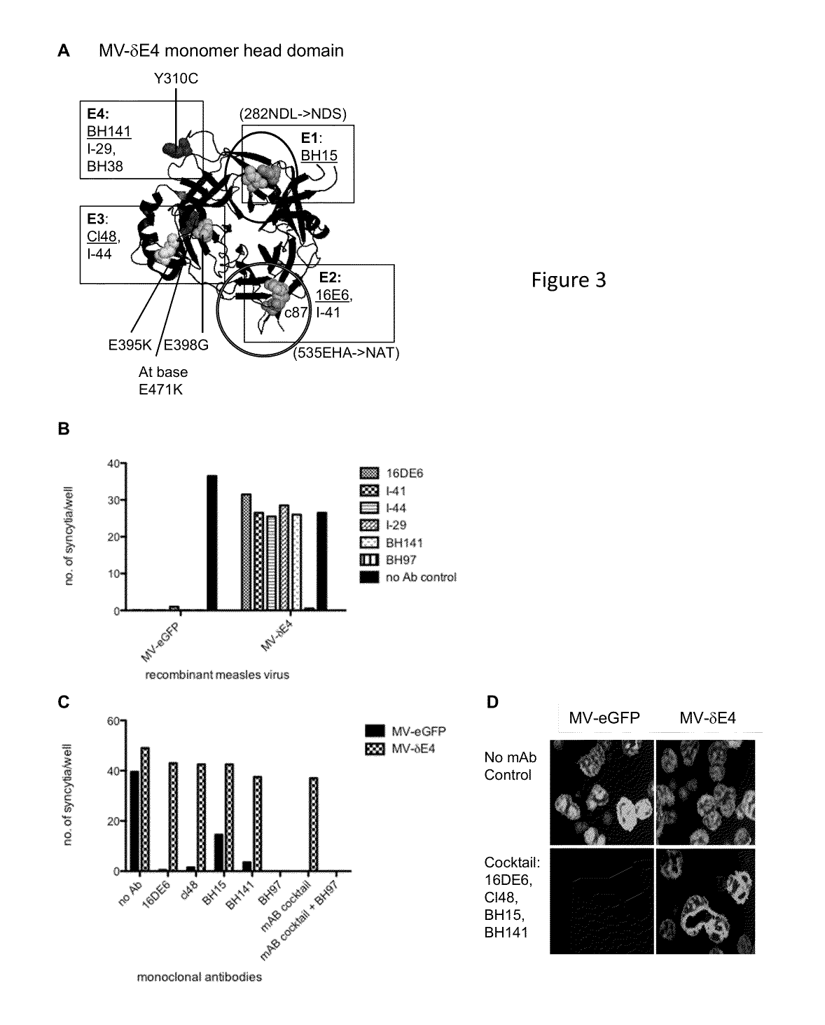

To protect the fourth and final epitope in this example, MV-δE3 was select in neutralizing concentration of BH38 recognizing E4. BH38 resistant clone, MV-δE4, encoded the escape mutation Y310C (FIG. 3A). This was the second time residue 310 has been implicated in the BH38 epitope. Previously, Y310D and L296I were identified as escape mutations in a BH38 escape MV variant (Bouche et al., Viral Immunol., 15:451-471 (2002)). Furthermore, Y310C in MV-δE4 provided resistance against other E4 targeting mAbs, BH141 and I-29 (FIG. 3B). BH141 and I-29 were used instead of BH38 in further neutralization assays to monitor resistance to E4.

MV-δE4 encoded escape mutations in four non-overlapping epitopes (E1-E4). Escape mutations included two glycosylated PNGS at N282 (E1) and N535 (E2) and point mutations E398G and E395K in (E3) and Y310C in (E4) (FIG. 3A). The following was performed to determine if MV-δE4 would escape neutralization by a cocktail of monoclonal antibodies targeting the four epitopes simultaneously. MV-δE4 was incubated in a mixture of one mAb from each of the four groups: BH15 (E1), 16DE6 (E2), c148 (E3), and BH141 (E4). BH97 was used as a control. MV-δE4 resisted neutralization by a cocktail of n-mAbs targeting E1-4 simultaneously, but was completely neutralized by control mAb BH97 (FIGS. 3C and 3D), for which it had no resistance.

### Escape Mutations in MV-δE4 Facilitate Viral Entry via CD46, SLAM, and Nectin-4, but Inhibit Cell-Cell Fusion via SLAM and Nectin-4

Binding of soluble H to receptors CD46 and SLAM can be completely or partially inhibited by 16DE6, I-29, and I-41 (E2, E4) and I-44 (E3), respectively (Santiago et al., J. Biol. Chem., 277:32294-32301 (2002)). The following was performed to determine the effect of escape mutations in MV-δE4 on viral entry via human CD46, SLAM, and Nectin-4.

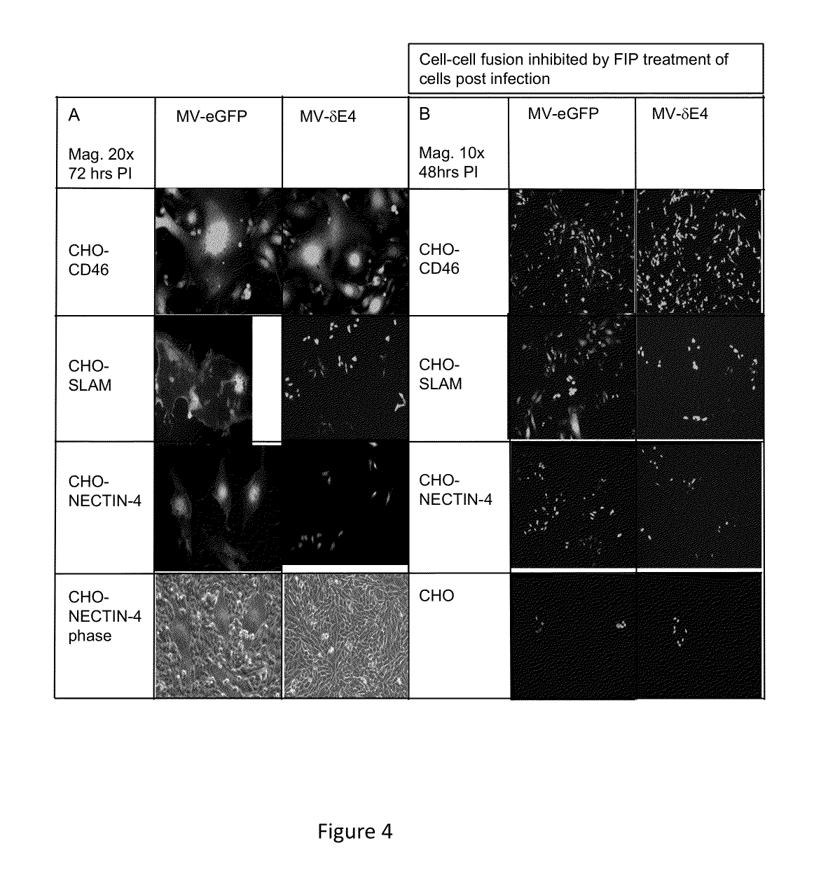

CHO cells can be infected with MV at very low levels via an unidentified receptor, but they do not support viral replication due to a cellular restriction. CHO, CHO-CD46, CHO-SLAM, and CHO-Nectin-4 cells were infected with MV-eGFP and MV-δE4 in the absence or presence of fusion-inhibitory-peptide (FIP) to monitor relative cell-cell fusion and infection of individual cells respectively (FIG. 4). MV-δE4 infected cells via all three receptors, but the escape mutations ablated fusion in CHO-SLAM, CHO-Nectin-4, but not CHO-CD46 cells. Loss of fusion also correlated to a moderately lower level of infection in these cells relative to cells infected with MV-eGFP.

### Example 2

### Engineering a Measles Vaccine with the H Polypeptide Modified with Glycan Shields and Escape Mutations for Early Vaccination of Infants in the Presence of Maternal Antibodies

### Cell Culture

Vero African green monkey kidney cells and Chinese hamster ovary (CHO) cells were grown in DMEM with 5% FBS. CHO-CD46 cells (Nakamura et al., Nat. Biotechnol., 22:331-336 (2004)) stably expressing human CD46 were maintained in DMEM, 10% FBS, 1 mg of G418 per mL. CHO-SLAM cells (Tatsuo et al., Nature, 406:893-897 (2000)) stably expressing human SLAM were grown in RPMI 1640, 10% FBS, 0.5 mg/mL of G418. CHO-Nectin-4 cells (obtained from Marc Lopez) stably expressing human Nectin-4 were maintained in RPMI 1640, 10% FCS, 0.5 mg/mL of G418, 0.1 mM MEM non-essential amino acids solution. All cell culture media were supplemented with 1% penicillin and streptomycin, and cells were grown at 37° C. in a humidified atmosphere of 5% CO₂.

### Site-Directed Mutagenesis of H.Shield

Mutations in H were performed in pCG-H (measles H glycoprotein (Hedmonston-tag strain) expression construct) using QuikChange® Site-Directed Mutagenesis Kit (Stratagene) as per manufacture's directions (Publication Number: 200518-12). Overlapping oligonucleotides were designed to introduce mutations in the H gene using pfuTurbo DNA polymerase (Stratagene). Following DpnI digestion of parental plasmid DNA, the synthetic DNAs were transfected into DH5a competent cells (Invitrogen). DNA sequence analysis was used to confirm the intended mutations and to ensure that the plasmids were free of unwanted second-site mutations. Following each round of mutagenesis, the mutated H gene was assayed for functionality in a measles fusion assay and designated a number, i.e. pCG-H42-H93.

### Measles Fusion Assay

After the mutant H genes were confirmed by sequencing, the mutations were tested for bioactivity using a cell fusion assay. 6-well plates were seeded with 4 mL of Vero cells (3×10⁶) in Dulbecco's Modified Eagle's Medium (DMEM, Lonza) supplemented with 5% Fetal Bovine Serum (FBS, Lonza) 24 hours in advance of the assay. The plates were incubated at 37° C. in a humidified environment containing 5% CO₂. The next day, the media was removed from the cells and replaced with 1 mL fresh, pre-warmed DMEM with 0% FBS. 2 μg of a plasmid encoding the measles fusion gene (F) was mixed with 2 μg of WT or mutant H DNA in a polystyrene tube, and pre-warmed DMEM-0% was added to a total volume of 50 μL. 7 μL of FuGene (Roche) was directly added to the DNA mixture, and the tubes were vortexed and incubated at room temperature for 15 minutes. The DNA-FuGene mixture was added to each well drop-wise and mixed by gently rocking the plate side to side. The plate was incubated at 37° C. for 30 minutes, and then rocked again and incubated at 37° C. for 5 hours. 2 mL of DMEM-5% was added to each well, rocked to mix, and returned to 37° C. to incubate. Plates were examined under a phase-contrast microscope to observe cell fusion over the next three days. Fused foci of cells (syncytia) were scored on a three point scale (+, +/−, and −).

### Plasmids, Virus Rescue, and Titration

To generate the full-length anti-genomic MV plasmids, in which the H glycoprotein was replaced by H#, p+MVeGFP and the pCG-H.Shield expression constructs were digested with PacI and SpeI, and H (Nse strain) was exchanged with the H.Shield fragments to create the plasmids p+MVeGFP-H.Shield (p+MV-H.Shield). Correct assembly of the constructs was verified by dideoxy-sequencing. MV was rescued, amplified, and tittered as described in Example 1.

### Establishing a Neutralizing Concentration of mAb

The ability of mAbs to inhibit MV-eGFP infection was assayed by the plaque reduction neutralization test (PRNT) in a 96 well format. mAbs were serially diluted in Opti-MEM (Invitrogen), and 50 μL was mixed with equal volume of MV-eGFP at 400 PFU/mL. The mixture was incubated for 1.5 hours at 37° C. before the addition of Vero cells (10,000 cells/well in 50 μL DMEM 5% FBS). Forty-eight hours post infection, the number of eGFP expressing syncytia/well were counted using a fluorescent microscope. The neutralizing concentration of mAb was the endpoint concentration of mAb required to reduce the number of MV-eGFP syncytia by 100%. Neutralization concentrations established for each mAb were as follows. 16DE6 (1.5 μL ascites/well), I-41 (1 μL ascites/well), I-44 (1.5 μL ascites/well), I-29 (6 μL ascites/well), BH141 (5 μL ascites/well), BH97 (2.5 μg/well), BH15 (3-6 μg/well) BH30 (1 μg/well), c148 (2 μg/well), c87 (50 μL hybridoma supernatant), c118 (50 μL hybridoma supernatant), c8 (50 μL hybridoma supernatant), 16CD-11 (1.5 μL ascites/well), BH18 (10 μg/well), BH47 (6 μL ascites/well), and 20H6 (50 μL hybridoma supernatant/well).

### Neutralization Assay

Neutralization assay was performed in a 96 well format. Each sample was duplicated, and the experiment was repeated 2 to 5 times depending on the mAb. Neutralizing concentration of mAb in 50 μL Opti-MEM or Opti-MEM alone (control) was mixed with equal volume of MV-eGFP or MV-H.Shield (25 PFU/well). In the case of non-purified mAb in hybridoma supernatant, 50 μL of hybridoma supernatant or 50 μL media were mixed with equal volume of virus. The mixture was incubated for 1.5 hours at 37° C. before the addition of Vero cells (10,000 cells/well in 50 μL DMEM 5% FBS). Forty-eight hours post infection, the number of eGFP expressing foci/well were counted using a fluorescent microscope.

### N-Linked Deglycosylation and Immunoblot Analysis

Viruses (2×10⁵ PFU) were incubated with and without PNGase F for 1 hour at 37° C. as per manufacturers protocol (New England Biolabs, P0705S). SDS loading buffer was then added to the samples, boiled for 5 minutes at 95° C., and separated on a 7.5% Tris-glycine SDS-polyacrylamide gel. Proteins were blotted to nitrocellulose membrane, immunoblotted with primary antibody (anti-rabbit anti-MV H protein (1:6000 dilution) (made by K. W. Peng, Mayo Clinic)), and secondary antibody (horseradish peroxidase-conjugated goat anti-rabbit (1:5000 dilution)).

### Structural Modeling

The crystal structure of measles virus hemagglutinin protein PDB ID: 3ALX was analyzed and manipulated using PyMOL software.

### Monoclonal Antibodies

Monoclonal antibodies used in this example were obtained from Drs. Claude Muller (Laboratoire National de Sante, Luxemburg; BH15, BH97, BH30, BH38, BH15, BH18, BH47, and BH141), Erling Norrby and Mariethe Ehnlund (Karolinska Institute, Sweden; 16DE6, I-41, I-44, I-29, and 16CD-11), Denis Gerlier (Inserm, France; c87 and c8), Branka Horvat (c118), Mark Federspiel (Mayo Clinic, USA; purified c148 but originally from T. Fabian Wild), and Ianko Iankov (Mayo Clinic, USA; 20H6).

### Infection of CHO Cells

CHO, CHO-CD46, CHO-Nectin-4, and CHO-SLAM cells were plated in a 96 well format and infected in triplicate. On the day of infection, media were removed from the cells, and 50 μL of virus were added in Opti-MEM (Invitrogen). Cells were infected with recombinant measles viruses at a moi of 0.1-0.8 (moi calculated based on TCID₅₀/mL as titered on Vero cells). Two hours post infection, 100 μL of media (cell type dependent) were added to the cells. Seventeen hours later, the media were replaced with fresh media or media with 200 nM fusion inhibitory peptide (FIP) (Bachem). Fluorescence microscopy was performed 48 and 72 hours post infection.

### Fluorescence Plaque Reduction Neutralization Assay

The PRNT was performed in a 96 well format where each sample was repeated in triplicate wells. Heat inactivated human AB serum (Valley Biomedicals, Inc. HS1017) was subjected to 2-fold serial dilutions (1:10 to 1:5120) in Opti-MEM. 50 μL/well of human serum dilution or Opti-MEM control (no serum) were incubated with 50 μL virus (MV-eGFP or MV.H.Shield at 25 PFU/well) for 2 hours or 24 hours at 37° C. and at 4° C. prior to infection of Vero cells (10,000 cells/well). Forty-five hours post infection, the number of eGFP expressing foci/well were counted. The 50% neutralizing dose (ND50, dilution of serum reducing the number of syncytia/infected foci by 50%) was calculated using Karber formula: log₁₀ ND₅₀=m−Δ(Σp−½). The full PRN titer of neutralization titer (NT) was the last serum dilution with no infection.

### Results

### Genetic Engineering of MV.H.Shield

The control virus used for all experiments in this example was MV-eGFP (Nse Strain encoding edmonston-tag strain H). MV-eGFP has a tropism for CD46, human SLAM, and Nectin-4. The H protein encoded by MV-eGFP was cloned into an expression plasmid, modified by site-directed mutagenesis, and cloned back into MV-eGFP to generate MV-H# mutants and MV.H.Shield.

The results from Example 1 were used as a foundation for the design of H.Shield that aimed to significantly shield the protein from recognition by polyclonal antibodies present in human serum. The escape mutations incorporated into H.Shield were designed to protect eight epitopes targeted by antibodies. Many of these were confirmed escape mutations when encoded independent of each other in MV-H# viruses screened in Table 2. The H gene in MV-eGFP was replaced with H. Shield, and the new vaccine prototype was named MV-H.Shield (Table 4).

TABLE 4
Mutations engineered into H.Shield of the MV-H.Shield vaccine
designed to protect seven epitopes.

| Potential immunodominant loops | Examples of PNGS | mAb escape |
|---|---|---|
| H236-250 | | |
| H261-269 | R261K | |
| H280-285 | 282NDL -> NDS | BH15 |
| H307-318 | | I-29, BH141, |
| | | BH30 |
| H403-407 | 403KDN -> NDT | Cl48, I-44 |
| H445-451 | N450Y | |
| H471-477 | 472WIP -> NIS | BH30 |
| H487-495 | 488KEA -> NAT | 16CD11 |
| H530-537 | 535EHA -> NAT | 16DE6, c87, |
| | | cl18, c8, I-41 |
| H552-562 | 552FYP -> NGT | C87, cl18, I-41 |
| H586-595 | 590SGG -> NGS | |

PNGS, potential N-linked glycosylation site. Defined by a sequon triplet of NXS/T, where X is any amino acid except for P.

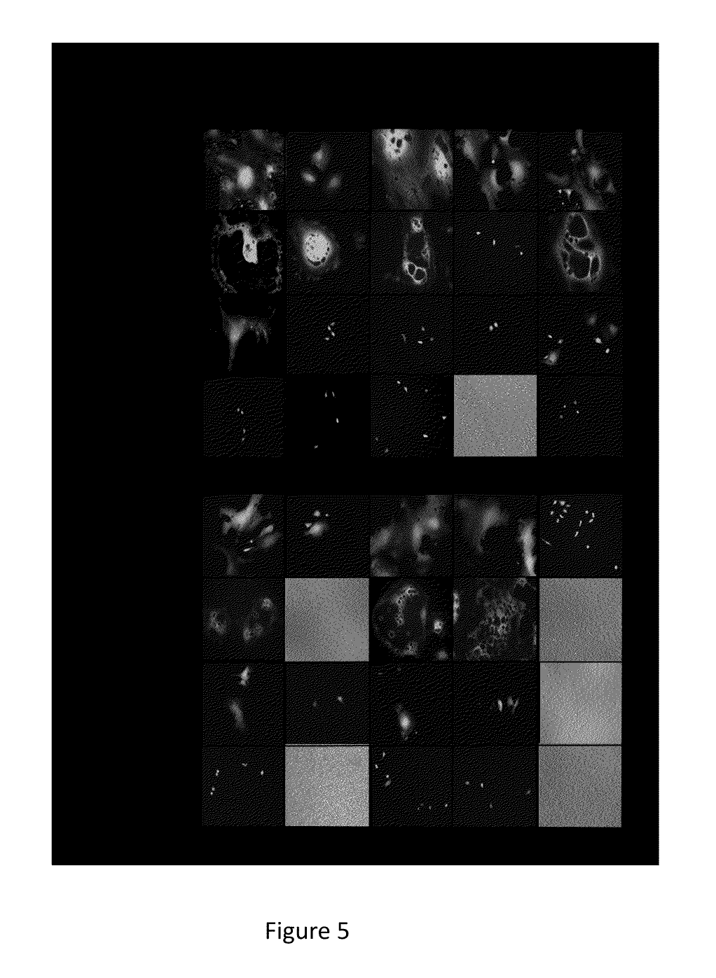

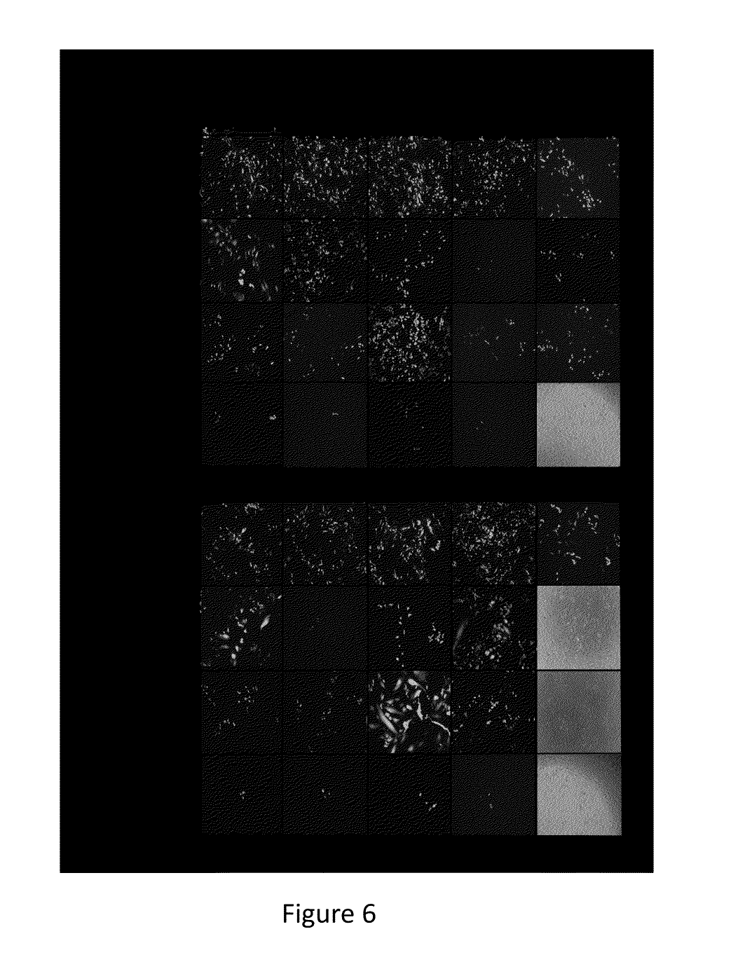

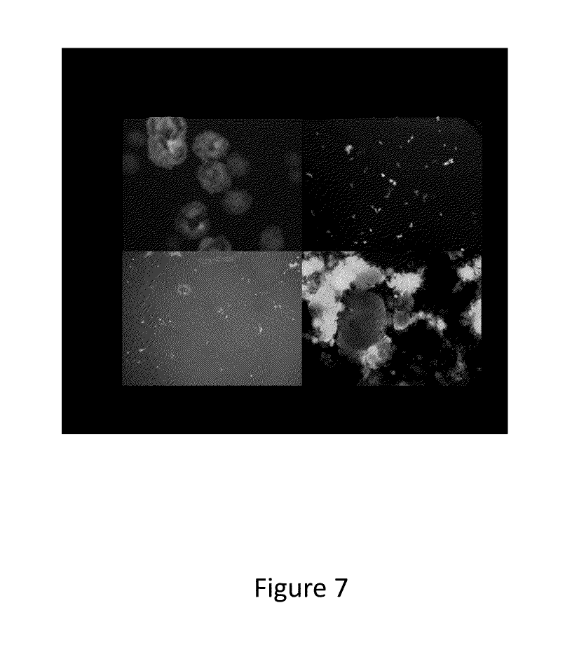

MV-H.Shield exhibited a significantly retarded fusion phenotype on Vero cells. Two days post infection, cell-cell fusion was rare (FIG. 7) and only started to become distinctly visible five days post infection (FIG. 7). Furthermore, MV-H.Shield lost tropism for Nectin-4 and SLAM (FIG. 5 and FIG. 6). The MV-H# mutants (Table 2) were used to identify most of the escape mutations in H.Shield responsible for the loss of SLAM and Nectin-4 tropism and fusion defects (FIG. 5 and FIG. 6). Infection was performed in CHO cells expressing human CD46, Nectin-4, and SLAM in the absence of presence of fusion inhibitory peptide (FIP) (FIG. 5 and FIG. 6). Treatment with FIP allowed for an empirical comparison of the relative number of infected cells forty-eight hours post-infection (FIG. 6). The major contributors to the CD46 tropic, fusion defective phenotype of MV-H.Shield were PNGS 535NAT and 552NGT (FIG. 5 and FIG. 6; MV-H11, MV-H21) and 488NAT.

MV-H-Shield-1 is Highly Glycosylated with N-Linked Glycans

MV-H.Shield encoded 12 PNGS (FIG. 8A). Five of these PNGS were native to H (H edmonston-tag—genotype A). Four of these PNGS were conserved amongst the genotypes and are glycosylated. Seven PNGS were rational designed to shield potential epitopes (listed in Table 4). Western-blotting of untreated and PNGase F treated MV-H# (Table 4), demonstrate N-linked glycosylation of 282NDL-→NDS, 535EHA→NAT and PNGS 403KDN→NDT and 552FYP∝NGT (FIG. 8B). The latter appeared to be only partially glycosylated, as indicated by the double band (FIG. 8; MV.H21). Santibanez and coworkers confirmed the glycosylation of PNGS 416DIS→NIS (Santibanez et al., J. Gen. Virol., 86:365-374 (2005)). This is a naturally occurring mutation in some Clade D genotypes.

PNGS 590SGG→NGS was not glycosylated (FIG. 8B; MV-H20). This PNGS was still incorporate into H.Shield on the basis of mutations flanking and within this sequon in genotypes B2 and D7, respectively (FIG. 3C).

### MV-H.Shield Escapes Monoclonal Antibodies Targeting E1-E6

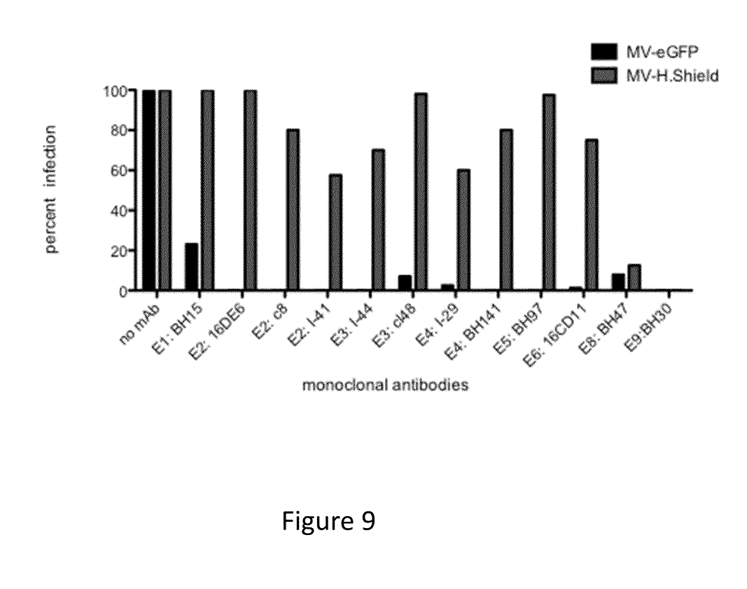

Many of the escape mutations combined in MV.H.Shield were screened individually or in 2-4 combinations for their ability to protect different epitopes against mAbs (Table 2 and Table 4). To confirm the success in protecting the same epitopes when the escape mutations were combined together, MV-H.Shield was challenged with mAbs that recognize E1-E6 (Table 4) in an in vitro neutralization assay (FIG. 9). Prior to infection of Vero cells, MV-H.Shield and MV-eGFP (control) were incubated with neutralizing concentration of mAbs for 1.5 hours at 37° C. Forty-eight hours later, infectious foci were quantified by counting the number of eGFP expressing foci per well. MV-H.Shield escaped complete neutralization by mAbs recognizing E1-E6 (FIG. 9). E7 in H. Shield was protected by PNGS 416NIS.

The epitope for BH30 was identified as H471-474. This was achieved by natural selection of MV in neutralizing concentrations of BH30. The clones selected encoded the mutations E471A or P474S. 472WIP→NIS PNGS was engineered into this epitope and found to protect MV from BH30 (Table 1). Sequence analysis of representative genotype strains identified a mutation in this epitope in genotype B3, C1, D4, D5.

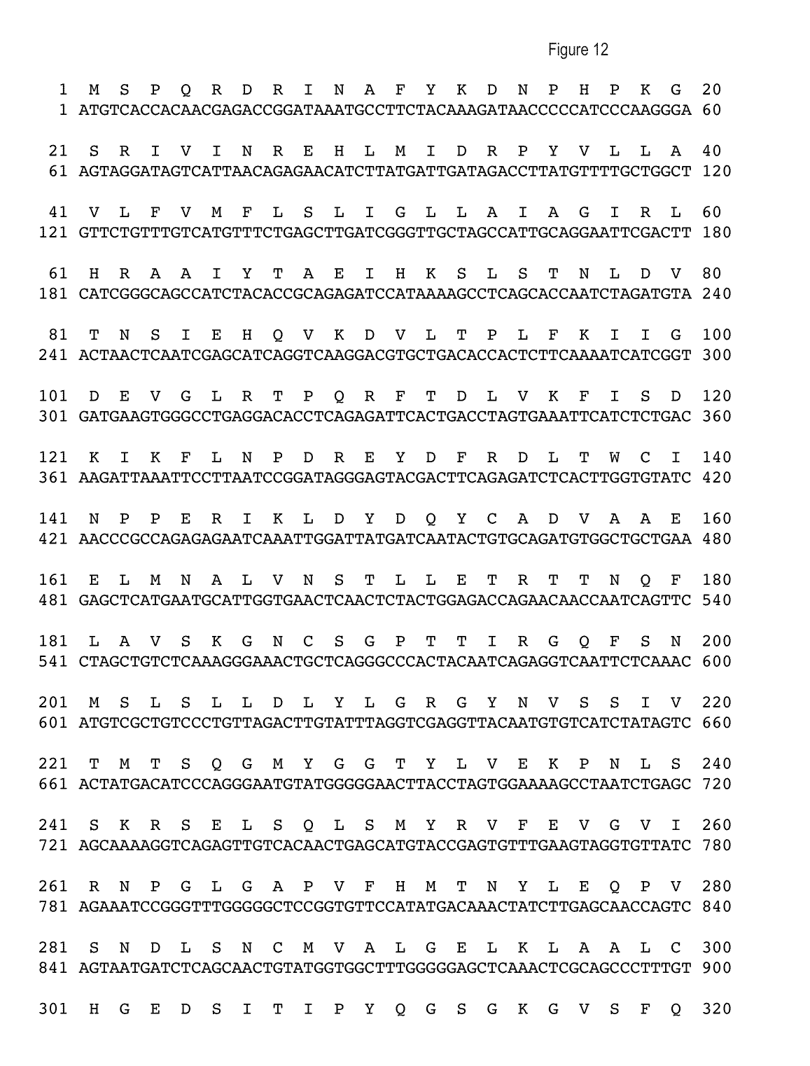

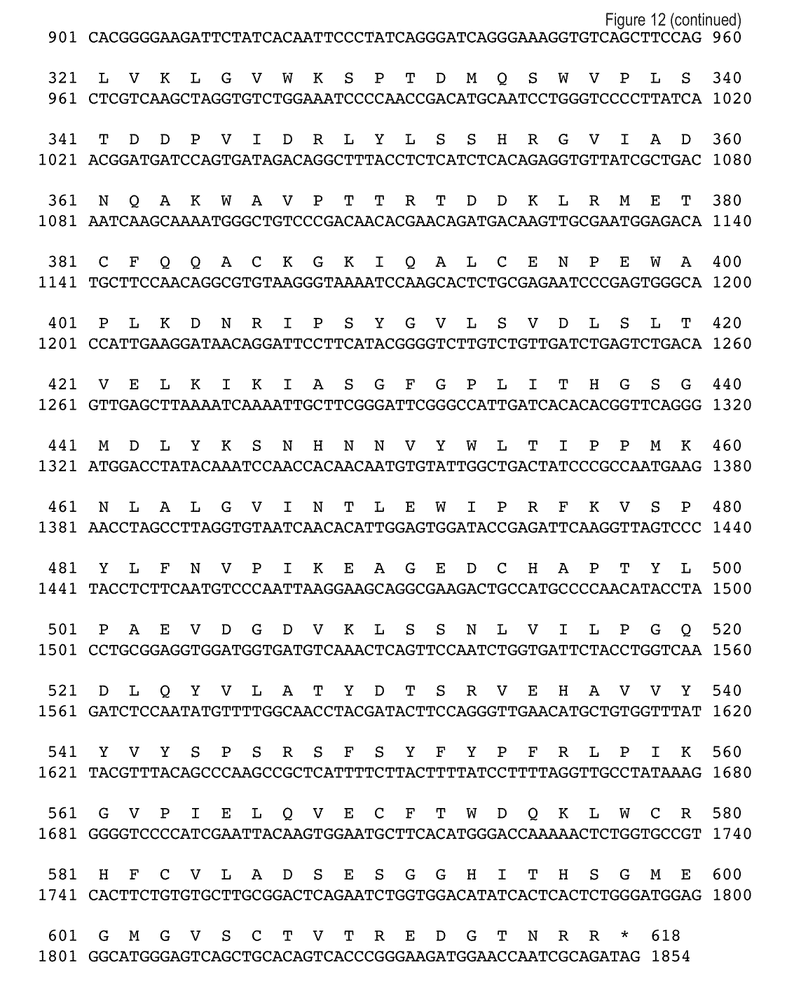

FIG. 12 provides the tetrameric structure of MV-H in complex with SLAM (form II), highlighting escape mutations in different eptiopes in H.Shield (Table 4) relative to receptor and dimer-of-dimers interface.

### MV-H.Shield-1 is Less Susceptible to Neutralization by Pooled Human Serum

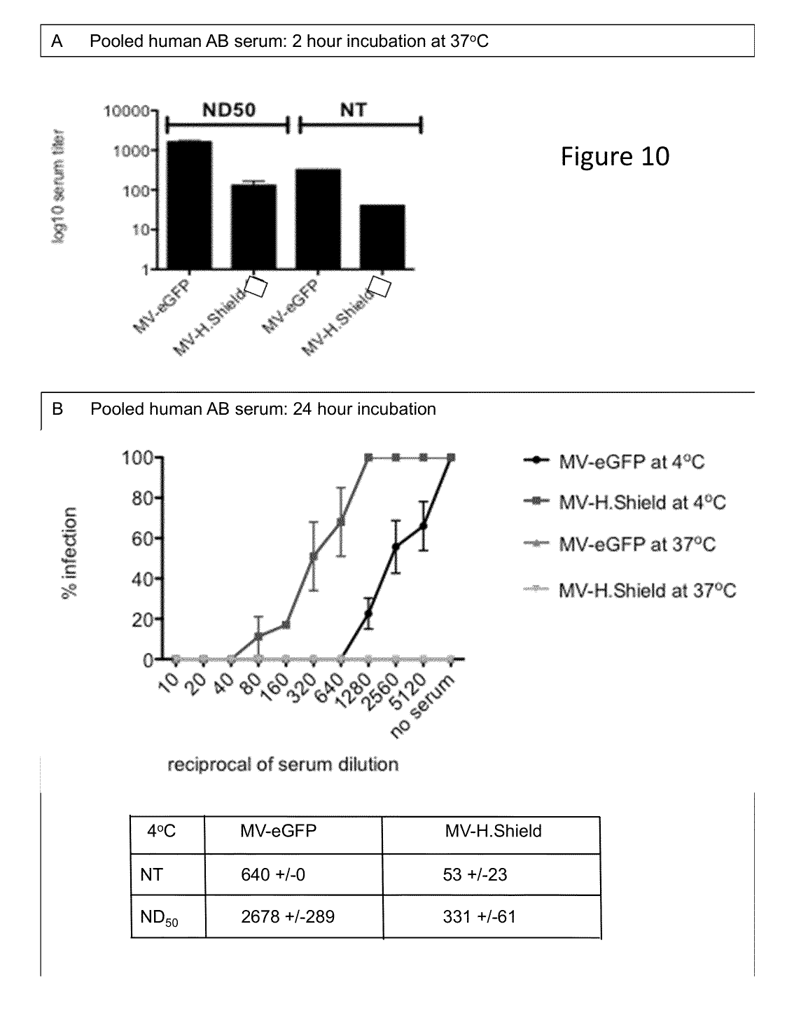

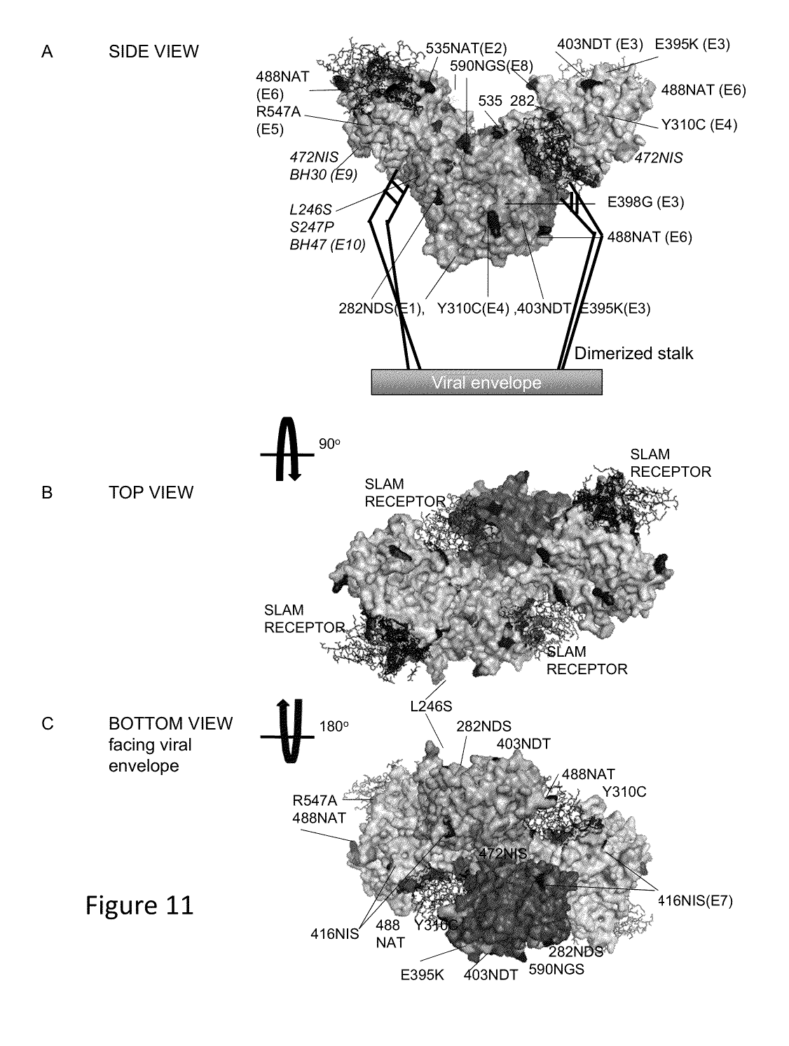

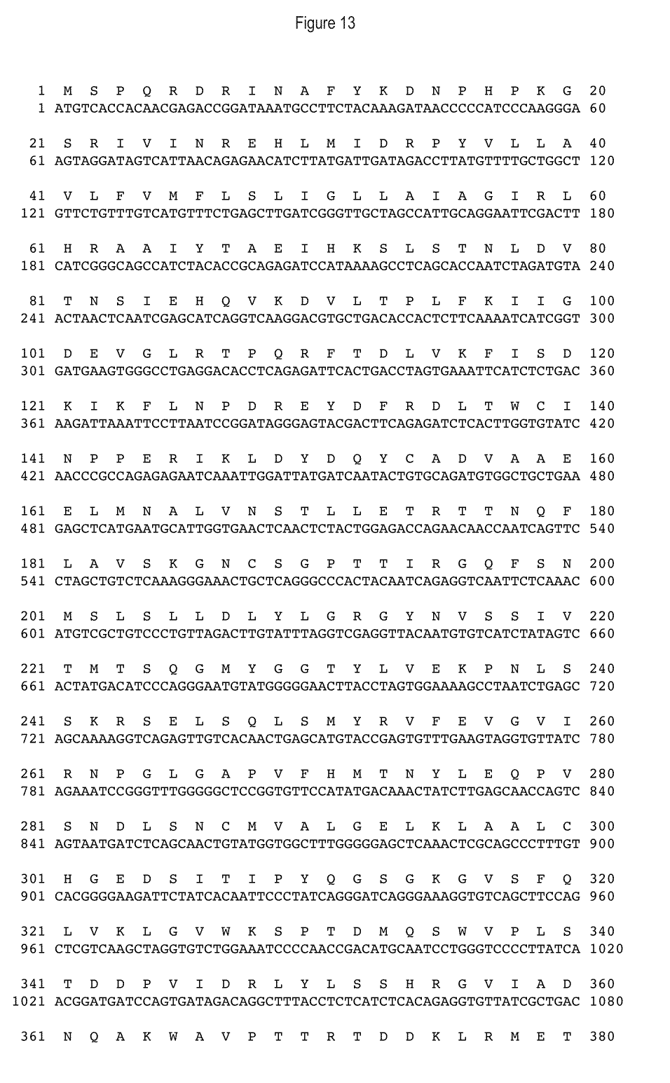

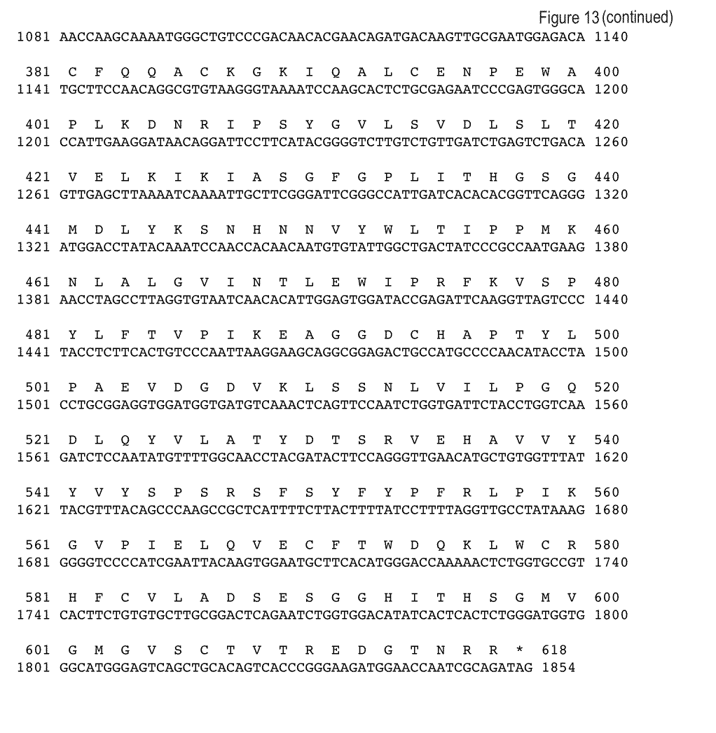

The level of measles virus-neutralizing antibody titers in commercially available pooled human serum was determined using a fluorescence-based plaque reduction micro-neutralization assay (based on plaque reduction neutralization test (PRNT). MV-H.Shield reproducibly infected cells at 8-10 fold higher serum dilutions than MV.eGFP, based on calculated serum ND₅₀ (serum dilution that reduces the number of plaques by 50%) and NT (serum dilution that reduces the number of plaques by 100%) (FIG. 10). To increase the chance of human antibodies binding to all available epitopes on MV, the time that viruses were incubated with human serum was increased from 2 hours (FIG. 10A) to 24 hours (FIG. 10B) at 37° C. and 4° C. (FIG. 10B). No infection of Vero cells by MV-eGFP or MV-H.Shield was seen in the absence or presence of serum, following a 24 hour incubation at 37° C. (FIG. 10B). When incubated at 4° C., the viruses were more stable, and once again MV-H.Shield infected Vero cells at 8-fold (ND₅₀) to 12 fold (NT) higher serum dilutions than MV-eGFP (FIG. 10B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A measles virus H polypeptide, wherein:
(i) said measles virus H polypeptide comprises the amino acid sequence set forth in SEQ ID NO:2 or SEQ ID NO:4 with the exception that said measles virus H polypeptide comprises an amino acid substitution at three or more of the positions selected from the group consisting of L284, Y310, E395, E398, K403, N405, D416, K488, E489, A490, E535, H536, A537, S546, R547, S550, F552, Y553, P554, S590, G592, W472, P474, and G316, or
(ii) measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, or 50 μL hybridoma supernatant of 20H6, when said measles viruses are placed into a well of a 96-well microtiter plate.

2. The measles virus H polypeptide of claim 1, wherein measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, or 50 μL hybridoma supernatant of 20H6, when said measles viruses are placed into a well of a 96-well microtiter plate.

3. The measles virus H polypeptide of claim 1, wherein measles viruses comprising said measles virus H polypeptide are not neutralized by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (l) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when said measles viruses are placed into a well of a 96-well microtiter plate.

4. The measles virus H polypeptide of claim 1, wherein measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, and 50 μL hybridoma supernatant of 20H6, when said measles viruses are placed into a well of a 96-well microtiter plate.

5. A nucleic acid encoding a measles virus H polypeptide, wherein:
(i) said measles virus H polypeptide comprises the amino acid sequence set forth in SEQ ID NO:2 or SEQ ID NO:4 with the exception that said measles virus H polypeptide comprises an amino acid substitution at three or more of the positions selected from the group consisting of L284, Y310, E395, E398, K403, N405, D416, K488, E489, A490, E535, H536, A537, S546, R547, S550, F552, Y553, P554, S590, G592, W472, P474, and G316;
(ii) measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 mg/well of BH18, 6 μL ascites/well of BH47, or 50 μL hybridoma supernatant of 20H6, when said measles viruses are placed into a well of a 96-well microtiter plate;
(iii) measles viruses comprising said measles virus H polypeptide are not neutralized by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (l) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when said measles viruses are placed into a well of a 96-well microtiter plate; or
(iv) measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, and 50 μL hybridoma supernatant of 20H6, when said measles viruses are placed into a well of a 96-well microtiter plate.

6. A recombinant virus comprising a measles virus H polypeptide and/or a nucleic acid encoding said measles virus H polypeptide, wherein:
(i) said measles virus H polypeptide comprises the amino acid sequence set forth in SEQ ID NO:2 or SEQ ID NO:4 with the exception that said measles virus H polypeptide comprises an amino acid substitution at three or more of the positions selected from the group consisting of L284, Y310, E395, E398, K403, N405, D416, K488, E489, A490, E535, H536, A537, S546, R547, S550, F552, Y553, P554, S590, G592, W472, P474, and G316;
(ii) measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL, ascites/well of BH47, or 50 μL hybridoma supernatant of 20H6, when said measles viruses are placed into a well of a 96-well microtiter plate;
(iii) measles viruses comprising said measles virus H polypeptide are not neutralized by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (l) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when said measles viruses are placed into a well of a 96-well microtiter plate;
(iv) measles viruses comprising said measles virus H polypeptide are not neutralized by 1.5 μL ascites/well of 16DE6, 1 μL ascites/well of I-41, 1.5 μL ascites/well of I-44, 6 μL ascites/well of I-29, 5 μL ascites/well of BH141, 1.5 μg/well of BH97, 3-6 μg/well of BH15, 1 μg/well of BH30, 2 μg/well of c148, 50 μL hybridoma supernatant of c87, 50 μL hybridoma supernatant of c118, 50 μL hybridoma supernatant of c8, 1.5 μL ascites/well of 16CD-11, 10 μg/well of BH18, 6 μL ascites/well of BH47, and 50 μL hybridoma supernatant of 20H6, when said measles viruses are placed into a well of a 96-well microtiter plate;
(v) said recombinant virus is a recombinant measles virus that is at least four times less sensitive than MV-Edm to neutralization by serum from a measles-vaccinated human; or
(vi) said recombinant virus is a recombinant measles virus that is at least four times less sensitive than MV-Edm to neutralization by at least three of the following: (a) 1.5 μL ascites/well of 16DE6, (b) 1 μL ascites/well of I-41, (c) 1.5 μL ascites/well of I-44, (d) 6 μL ascites/well of I-29, (e) 5 μL ascites/well of BH141, (f) 1.5 μg/well of BH97, (g) 3-6 μg/well of BH15, (h) 1 μg/well of BH30, (i) 2 μg/well of c148, (j) 50 μL hybridoma supernatant of c87, (k) 50 μL hybridoma supernatant of c118, (1) 50 μL hybridoma supernatant of c8, (m) 1.5 μL ascites/well of 16CD-11, (n) 10 μg/well of BH18, (o) 6 μL ascites/well of BH47, and (p) 50 μL hybridoma supernatant of 20H6, when said measles virus is placed into a well of a 96-well microtiter plate.

7. The recombinant virus of claim 6, wherein said virus is a measles virus.


SEQUENCE LISTING (4 sequences)

SEQ ID NO: 1 - DNA, 1854 bp, Measles virus
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggaattcgacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcaatgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggagggcatgggagtcagctgcacagtcacccgggaagatggaaccaatcgcagatag

SEQ ID NO: 2 - protein, 597 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 3 - DNA, 1854 bp, Measles virus
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggaattcgacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaaccaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcggagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatag

SEQ ID NO: 4 - protein, 614 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMNLALGVINTLEWIPRFKVSPYLFTVPIKEAGGDCHAPTYLPAEVDGDVKLSSNLVILPGDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR